(19) United States
(12) Patent Application Publication
(10) Pub. No.: US 2025/0268937 A1
(43) Pub. Date: Aug. 28, 2025

(54) ENGINEERED IMMUNE CELLS TO TARGET SARS-COV2

(71) Applicant: The Johns Hopkins University, Baltimore, MD (US)

(72) Inventors: Challice Bonifant, Baltimore, MD (US); Ilias Christodoulou, Baltimore, MD (US)

(73) Assignee: The Johns Hopkins University, Baltimore, MD (US)

(21) Appl. No.: 18/029,592

(22) PCT Filed: Sep. 30, 2021
(86) PCT No.: PCT/US2021/053042

§ 371 Date: Mar. 30, 2023

Related U.S. Application Data
(60) Provisional application No. 63/085,937, filed on Sep. 30, 2020.

(51) Int. Cl. A61K 35/17; A61K 40/11; A61K 40/15; A61K 40/31; A61K 40/48; A61P 31/14; C07K 16/16

(52) U.S. Cl. CPC A61K 35/17; A61K 40/11; A61K 40/15; A61K 40/31; A61K 40/48; A61P 31/14; C07K 16/16; A61K 2239/13

(57) ABSTRACT

Provided herein are, inter alia, methods, compositions and kits for treating or preventing coronaviruses, e.g., SARS-CoV-2. Also included herein are kits for treating or preventing coronaviruses, e.g., SARS-CoV-2.

The present application claims the benefit of U.S. Provisional Patent Application No. 63/085,938 filed Sep. 30, 2020, which is incorporated by reference herein.

### BACKGROUND

New compositions and methods for treating coronaviruses, specifically SARS-CoV-2 (severe acute respiratory syndrome coronavirus 2) are needed.

### BRIEF SUMMARY

Provided herein are, inter alia, methods, compositions and kits for treating and preventing coronaviruses, e.g., SARS-CoV-2. Also included herein are kits for treating coronaviruses, e.g., SARS-CoV-2.

More particularly, in a first aspect, engineered immune cell are provided that can express a chimeric antigen receptor (CAR) polypeptide comprising an extracellular banana lectin (BanLec) domain.

In one preferred aspect, the BanLc domain comprises an acid sequence having an H84T substitution (i.e. replacing histidine 84 with a threonine).

Preferred cells include those that express a chimeric antigen receptor (CAR) polypeptide comprising an extracellular banana lectin (BanLec) domain, wherein the BanLec domain comprises the amino acid sequence having at least 70, 75 80, 85, 90, 92, 93, 94, 95, 96, 97, 98, 99 or 100% identity with an amino acid sequence of SEQ ID NO: 3 or a fragment thereof, and/or wherein the BanLec domain is encoded by a polynucleotide sequence having at least 70, 75, 80, 85, 90, 92, 93, 94, 95, 96, 97, 98, 99 or 100% identity with the nucleic acid sequence of SEQ ID NO: 4.

Further preferred are engineered immune cells include those where the CAR polypeptide comprises the amino acid sequence having at least 70, 75, 80, 85, 90, 92, 93, 94, 95, 96, 97, 98, 99 or 100% identity with an amino acid sequence of SEQ ID NO: 6 OR SEQ ID NO: 7, and/or wherein the CAR polypeptide is encoded by a sequence that has at least 70, 75, 80, 85, 90, 92, 93, 94, 95, 96, 97, 98, 99 or 100% identity with SEQ ID NO: 8 or SEQ ID NO: 10.

In certain aspects, the immune cells may comprise a natural killer (NK) cell, a T-cell, a B-cell, a macrophage or other myeloid lineage cell, or a mesenchymal stem cell.

In additional aspects, methods are provided for preventing or treating a viral infection, the method comprising: administering to a subject a composition comprising a population of engineered immune cells as described herein expressing a chimeric antigen receptor (CAR) polypeptide comprising an extracellular banana lectin (BanLec) domain, wherein the BanLec domain comprises an amino acid sequence having at least 70, 80, 85, 90, 92, 93, 94, 95, 96, 97, 98, 99 or 100% identity with an amino acid sequence of SEQ ID NO: 3 or a fragment thereof, thereby preventing or treating the viral infection.

In certain aspects, the expressed CAR polypeptide of the cells has at least 70, 75, 80, 85, 90, 92, 93, 94, 95, 96, 97, 98, 99 or 100% identity of SEQ ID NO: 4 or SEQ ID NO: 6.

In certain aspects, the viral infection may be a coronavirus infection, including severe acute respiratory syndrome coronavirus 2 (SARS-CoV-2).

The administered population of engineered immune cells suitably may be autologous or allogenic to the subject.

In certain aspects, the population of engineered immune cells are frozen prior to administrating to the subject.

In preferred methods, one or more additional, distinct antiviral agents can be administered to subject.

In a particular aspect, a chimeric antigen receptor (CAR) that incorporates H84T-BanLec (Banana Lectin (BanLec) that has a H84T point mutation) as the extracellular moiety is provided. In one aspect, a preferred banana lectin (BanLec) has a single point mutation, particularly at position 84 from histidine to threonine, which may be referred to as H84T BanLec,

In one aspect, a H84T-BanLec CAR as provided herein can specifically direct NK cell binding of SARS-CoV-2 envelope glycosites to promote viral clearance. In particular aspects, the H84T-BanLec CAR can be stably expressed at high density such as on primary human NK cells during ex vivo expansion. Preferred H84T-BanLec CAR-NK cells can inhibit viral entry of S-protein pseudotyped lentivirus into 293T cells expressing ACE2, the receptor for SARS-CoV-2. NK cells can be activated to secrete inflammatory cytokines when in culture with virally infected cells. The present H84T-BanLec CAR-NK cells therefore can be an effective cell therapy for patients suffering from COVID-19.

Kits are also provided that suitably may include 1) a population of engineered immune cells expressing a chimeric antigen receptor (CAR) polypeptide comprising an extracellular banana lectin (BanLec) domain and optionally 2) instructions for therapeutic use of the cells, such as to treat a subject suffering or suspected of a coronavirus infection.

Other aspects of the invention are disclosed infra.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a schematic of CAR-NK targeting of SARS-COV2 and infected human cells. The schematic depicts that engineered H84T-BanLec. CAR NK cells specifically bind viral envelope and virus-infected cells.

FIGS. 2A-2C are data depicting that 2B4.ζ and e 41BB.z cells are activated by AML targets. FIG. 2A are histogram plots showing the transduction efficiency of NK-CARs. Flow cytometric detection using rhCD123-His and αHis-APC was used. Isotype: blue, CAR-NK, purple/pink. FIG. 2B is a bar graph showing NK activation measured with co-culture and ELISA for IFNγ secretion. Raji (CD123−), and MV4-11 (CD123+) used as target cells. Bar height represents percent change of IFNγ present in supernatant of co-culture as compared to cells cultured in the absence of target. FIG. 2C is a bar graph showing cytotoxicity evaluated in 72 hr co-culture assays using FACS-based cell counting. Raji Effector:Target cell ratio 1:1, MV-4-11 E:T as noted.

FIGS. 3A and 3B are data showing that the H84T-BanLec CAR can be engineered for T cell surface expression and that H84-BanLec CAR T cells are specific for lung cancer. FIG. 3A depict images of blots showing the detection of CAR. Western blot performed with H84T-BanLec antibody. GAPDH used as loading control. FIG. 3B is a graph showing the cytotoxicity of H84T-BanLec CAR T cells to A549 lung cancer cell line. NT (non-transduced) T cells and MRC5 normal lung fibroblast cell line used as controls. Experiments were performed with Incucyte imager using 4 images per well, 3 wells per condition, effector:target ratio of 1:1. The NT T cells are the same T cell population as the CAR, but without CAR expression.

FIG. 4 is a schematic of CAR-NK cell production. i.) Peripheral Blood Mononuclear Cells (PBMCs) isolated from donor peripheral blood. ii.) T cells depleted iii.) NK cells stimulated with lethally irradiated feeder cells iv.) NK cells maintained and expanded in recombinant IL-2 (interleukin 2), v.) NK cells transduced with replication incompetent retroviral vector carrying CAR coding sequences.

FIG. 5 is an image of a blot showing H84T Ban-Lec CAR expression in human NK cells, detected via Western blot against the TCRζ chain (C-terminal component of CAR), NT: non-transduced NK cells included as negative control. 2B4 and 41BB refer to intracellular signaling components encoded by unique H84T BanLec CAR constructs.

FIGS. 6A and 6B are images showing infectivity of S-protein pseudotyped lentivirus to cells expressing the human ACE2 receptor. FIG. 6A are histogram plots showing data depicting 293T cell line engineered for hACE expression. Grey: Parental cells, Red: 293T.hACE2. FIG. 6B is a bar graph showing infectivity of replication incompetent retrovirus carrying human firefly Luciferase pseudotyped with the SARS-CoV-2 Spike protein. This viral vector was used to transduce 293T and 293T.hACE2 cells at different viral titers (neat, 1:5, 1:25, 1:125). Viral entry measured by detection of bioluminescence.

FIG. 7 (includes FIGS. 7A-7E). H84T-BanLec.4-1BB.ζ CAR expression in human NK cells. FIG. 7A. Schema defining CAR components. FIG. 7B. Quantification of retroviral vector copy number (VCN) in transduced NK cells (CAR). Untransduced/unmodified (UTD) NK cells served as negative controls. n=4 NK cell donors FIG. 7C. Western blot detection of protein. UTD: untransduced NK cell lysate, CAR: H84T-BanLec CAR-NK cell lysate. Blue arrows: CAR, red arrows: endogenous zeta chain. GAPDH was used as a loading control. FUG. 7D. Representative histogram showing detection of CAR-expression with flow cytometry. Gray: UTD, Blue: CAR-NK. FIG. 7E. CAR detection on primary NK cell surface on days 4 and 14 post-transduction. Each dot representative of single transduction. n=6 total replicates from 4 independent NK cell donors.

FIG. 8 (includes FIGS. 8A-8C). Recombinant SARS-CoV-2 proteins bind hACE2.293T. FIG. 8A. Schema of 293T engineered with hACE2. FIG. 8B. Flow cytometric analysis of 293T expressing hACE2. FIG. 8C. Detection of recombinant SARS-CoV-2 spike (S)-proteins bound to hACE2-expressing 293T cells. 293T without hACE2 expression used as negative control.

FIG. 9 (includes FIGS. 9A-9E). CAR-NK cells decrease infectivity of S-protein pseudotyped virus. FIG. 9A. Schema of SARS-CoV-2 pseudovirus infection of hACE2.293T. Pseudoviral particles contain plasmids encoding ffLuc. Following viral entry, cells emit bioluminescence (BL) after D-Luciferin metabolism. FIG. 9B. Measurement of target cell BL emission following transduction with S-protein pseudotyped virus carrying ffLuc reporter gene. Assay performed in triplicate. FIG. 9C. Schematic representation of the BanLec-CAR NK cells blocking hACE2.293T infection. FIG. 9D. NK cells plated with target cells (hACE2.293T) and pseudovirus at indicated effector to target (E:T) ratios. Percent infectivity calculated using condition with 293T.ACE2 and pseudovirus alone. (E:T=0.4, p=0.03; E:T=1, p=0.05, n=6, 2 separate experiments using 3 independent NK cell donors, each experiment performed in triplicate). FIG. 9E. Quantification (pg/mL) of IFNγ and TNFα present in culture media of NK cells at baseline and in co-culture with S-pseudotyped virus infected hACE2.293T (black: no target, red: co-culture: n=3 donors: Mean value+/−SEM; baseline vs. co-culture *p<0.05, ***p<0).

FIG. 10 (includes FIGS. 10A-10C). Unmodified and BanLec CAR-NK cells are equally cytotoxic against hACE2.293T targets in the absence of pseudovirus. FIG. 10A. Schema of 293T engineered with both hACE2 and firely Luciferase (ffLuc). FIG. 10B. Representative pseudocolor plots of 293T, hACE2.293T, 293T.ffLuc and hACE2.293T.ffLuc showing ffLuc and hACE2 expression. FIG. 10C. NK cells were co-cultured at indicated E:T ratios with hACE2.293T.ffLuc.Bioluminescence (BL) measured following addition of D-luciferin and compared to control condition without effector cells as an indicator of target cell death (n=6, 2 separate experiments of 3 independent NK cell donors, each experiment performed in triplicate).

### DETAILED DESCRIPTION

Provided herein are, inter alia, methods, compositions and kits for treating and preventing coronaviruses, e.g., SARS-CoV-2.

SARS-CoV-2 is the virus responsible for the COVID-19 global pandemic. H84T-BanLec is a lectin that specifically binds viral glycoproteins, including those of SARS-CoV-2. Though H84T-BanLec a promising antiviral agent, pharmaceutical development is hindered by predicted in vivo chemical and biological degradation. In addition, critically ill patients have immunodysfunction likely to prevent complete virus eradication, even if BanLec binding diminishes infectivity. Provided herein is the use of the binding properties of H84T-BanLec to stimulate powerful NK-cell anti-SARS-CoV-2 activity. Healthy NK (natural killer) cells are genetically engineered to express a receptor coupling glycoprotein recognition with NK activation. T cells were engineered to express a chimeric antigen receptor (CAR) with an H84T-BanLec extracellular domain (BanLec-CAR). BanLec-CAR T cells demonstrates antigen-specific activation and cytotoxicity. However, when adoptively transferred, donor T cells carry the risk of severe graft-versus-host disease. NK cells are an alternate immune effector cell with a critical role in the clearance of viral infections. The infusion of allogeneic donor NK cells has been shown safe in a number of clinical trials.

BanLec CAR-NK cells are used to target SARS-CoV-2 virus and virally infected cells. H84T-BanLec.CAR NK cells are generated. The binding to SARS-CoV-2, specific NK-cell activation, and cytotoxicity against infected cells decorated with SARS-CoV-2 envelope glycoproteins is evaluated. H84T-BanLec.CAR NK cells are tested in animal models of SARS-CoV-2 infection. These cells may represent an off-the-shelf cell therapy for patients suffering from COVID-19 or other coronaviruses.

### Significance and Advantages of the Invention

Since late December 2019, SARS-CoV-2, a novel betacoronavirus, emerged in Wuhan, China and has spread rapidly across the globe. COVID-19, the respiratory infection caused by this virus, has thus far killed over 999,000 and infected over 33.2 million people throughout the world, resulting in one of the deadliest pandemics humanity has ever faced. COVID-19 infection can be severe, with resultant respiratory failure and death. At present, no drug or therapy has been effective in clearance of the viral infection and afflicted patients receive only symptomatic management. The toll on humanity has been enormous. In addition to the disease itself, preventive measures have profoundly altered societal norms and disrupted the global economy. Thus, there is a great need for an effective treatment.

Those critically ill with COVID-19 exhibit immune dysregulation that impairs their capability to clear severe infection. Patients with COVID-19 pneumonia or respiratory failure are lymphopenic with profoundly decreased levels of B, CD4+T, CD8+T, and NK cells. The degree of observed lymphopenia correlates with disease severity. Transfer of healthy donor immune cells to these patients is therefore a strategy with promise. Both T and NK cells are immune effectors with important biological functions in the clearance of pathogens. Acute deterioration with COVID-19 requires emergency treatment options available at the ready.

In comparison to T cells, which must be manufactured from autologous cells in order to prevent graft-versus-host disease, NK cells can be infused from allogeneic donors without this risk. Because of this, a bank of cells collected from healthy donors and frozen can be stored as an off-the-shelf cellular product. These would then be readily available for infusion when needed.

NK cells can be isolated from healthy donors' peripheral blood and expanded ˜300-fold ex-vivo after 3 weeks. Once activated, NK cells can then be genetically engineered to specifically target surface antigens using chimeric antigen receptors (CARs). Aliquots of manufactured CAR-NK cells can then be frozen, to establish a master cell bank capable of treating patients suffering from COVID-19 and in need of adequate immune function. Indeed, there are open clinical trials in China investigating the potential role of NK cell (clinicaltrials.gov: NCT04280224) and CAR-NK cell (clinicaltrials.gov: NCT04324996) adoptive transfer as COVID-19 treatment. Expression of a CAR on the surface of NK cells can potentiate enhanced antigen-specific activation and target killing. This boosted function may render CAR-NK cells superior effectors in clearing circulating virus and virally infected cells. Optimal CAR design is essential in modifying NK cell behavior. Appropriate antigen targeting as well as a precise combination of intracellular signaling domains are critical.

Effective transmembrane and intracellular CAR components for NK cell activation were identified. Moreover, instead of targeting a specific antigenic peptide, glycosylation patterns common to viral envelopes are targeted. Decreased expression of target peptides is a mechanism employed by cancer cells to evade targeted immunotherapies. The flexibility in viral targeting diminishes the potential risk of antigen downregulation. At the same time, the specificity in recognition of virally infected cells and viral particles is maintained.

The H84T-BanLec. CAR NK cells are used as specific binders of the SARS-CoV-2 viral envelope. These engineered cells not only bind and eliminate circulating virus, but also clear infected respiratory epithelial cells. Animal testing and further therapeutic development is performed. Given the paucity of treatments available for patients suffering from COVID-19, a cell therapy product providing effector cell function with exquisite antiviral specificity would have profound impact.

### BanLec (Banana Lectin)

BanLec is a member of the mannose-specific jacalin-related lectin (mJRL) group that functions as a potent T-cell mitogen. It forms a dimer with two carbohydrate-binding sites (CBS I and CBS II) in each protein subunit. BanLec associates with high-mannose-type N-glycans on the HIV-1 envelope and can thus block viral entry into cells. BanLec has a number of similarities to Concanavalin A and binds to mannose-related carbohydrate structures. It has highly immunogenic properties, including, for example that it induces a strong IgG4 antibody response, and appears to be an important antigen involved in banana allergies.

### BanLec Binding of Novel Coronavirus

BanLec is a lectin extracted from the fruit of bananas (Musa acuminate) that binds high mannose glycans. The novel coronavirus, SARS-CoV-2, mediates cell entry via association of its trimeric spike protein with the human Angiotensin-converting enzyme 2 (ACE2) receptor. The spike protein is decorated with underprocessed oligomannose, as is common in other viruses. In the case of SARS-CoV-2, virus-specific high mannose glycosites are in proximity and shield the receptor binding site of the spike protein. They are therefore not likely to be mutagenic hotspots. Notably, BanLec has been experimentally determined to bind HIV, influenza, and other coronaviruses. This binding has an antiviral effect, but wild-type BanLec is also strongly mitogenic and induces unspecific T cell activation. BanLec mitogenicity can be divorced from antiviral activity via a point mutation. A single amino acid substitution (H84T), retains the binding capacity of this BanLec to monosaccharides (responsible for glycoprotein binding), but diminishes the multivalent interactions that drive T-cell activation. Importantly, H84T-BanLec has been shown to inhibit SARS-CoV-2 viral infectivity when tested using the highly susceptible African green monkey Vero-E6 cell line.

An exemplary BanLec amino acid sequence is publically available at the NCBI database under accession number 2BN0_A, incorporated herein by reference in its entirety (SEQ ID NO: 1). The amino acid, H84, is bold and underlined.

| | |
|---|---|
| 1 | mngaikvgaw ggnggsafdm gpayriisvk ifsgdvvdgv dvtftyygkt etrhyggsgg |
| 61 | tpheivlqeg eylvgmagev anyhgavvlg klgfstnkka ygpfgntggt pfslpiaagk |
| 121 | isgffgrggk fldaigvyle p |

An exemplary BanLec nucleic acid sequence is publically available at the NCBI database under accession number: AY103481.1, incorporated hereby reference in its entirety (SEQ ID NO: 2). The start and stop codons are bold and underlined.

| | |
|---|---|
| 1 | atgaacggag cgatcaaggt gggagcatgg ggagggaacg gagggtcggc cttcgacatg |
| 61 | ggacctgctt atcgtatcat cagcgtcaag attttttccg gagacgtggt cgacgccgtg |
| 121 | gacgtcacct tcacctacta cgggaagacg gagacccgac acttcggtgg cagcggtggt |
| 181 | actccccacg aggtttgcat cactaccaat ctcaaagctc atagctgact gcagattaat |
| 241 | ggcttctact tggatgcaga ttgttctgca ggagggcgag tatctggtgg gaatgaaggg |
| 301 | agaatttggt aactaccatg gagtggtggt ggtggggaag cttggcttca gcaccaacaa |
| 361 | gaaatcctac ggacctttcg gcaacacggg agggactccc ttctcccttc ctatagcagc |
| 421 | aggcaagatc tctggcttct tcggccgtgg cggcgatttt attgacgcca ttggggtcta |
| 481 | cttggagcca taattggcca ctgcagtaaa tcacaagagt tgctatgtgc tacttggagt |
| 541 | gatgagatga agaatgtctg caataaatgg atcgg |

An exemplary BanLec amino acid sequence comprising a single amino acid substitution (H84T) that retains binding capacity to monosaccharides, responsible for glycoprotein binding, but diminishes the multivalent interactions that drive T-cell activation is provided below (SEQ ID NO: 3). H84T is bold and underlined.

| | |
|---|---|
| 1 | mngaikvgaw ggnggsafdm gpayriisvk ifsgdvvdgv dvtftyygkt etrhyggsgg |
| 61 | tpheivlqeg eylvgmagev anytgavvlg klgfstnkka ygpfgntggt pfslpiaagk |
| 121 | isgffgrggk fldaigvyle p |

An H84T BanLec nucleic acid sequence is provided as follows (SEQ ID NO: 4):

AATGGCGCTATCAAAGTTGGAGCCTGGGGCGGCAATGGCGGCAGCGCTT
TTGATATGGGCCCTGCCTACCGGATCATCAGCGTGAAGATCTTTAGCGG
CGACGTGGTGGATGGCGTGGACGTGACCTTTACCTACTACGGCAAGACC
GAGACACGGCACTATGGCGGAAGCGGAGGAACACCTCACGAGATCGTTC
TGCAAGAGGGCGAGTACCTCGTTGGAATGGCTGGCGAGGTGGCCAACTA
TACAGGTGCTGTGGTGCTGGGCAAGCTGGGCTTCAGCACCAACAAGAAG
GCCTACGGACCCTTCGGCAATACCGGCGGCACACCTTTTAGCCTGCCTA
TTGCCGCCGGAATCAGCGGCTTTTTTGGCAGAGGCGGCAAGTTCCTGGA
TGCCATCGGAGTGTATCTGGAACCC

An H84T BanLec amino acid sequence is provided as follows (SEQ ID NO: 5):

NGAIKVGAWGGNGGSAFDMGPAYRIISVKIFSGDVVDGVDVTFTYYGKT
ETRHYGGSGGTPHEIVLQEGEYLVGMAGEVANYTGAVVLGKLGFSTNKK
AYGPFGNTGGTPFSLPIAAGISGFFGRGGKFLDAIGVYLEP

### NK (Natural Killer) Cells in Viral Infections

NK cells are innate lymphocytes that are important in the control of viral infections. NK cell activation and resultant cytotoxicity is regulated by the interplay of inhibitory and activating receptors. Virally infected cells downregulate the expression of MHC class I molecules, the ligands of NK cell inhibitory receptors. In addition, virus-derived products and stress-induced ligands are expressed on infected cells. These can stimulate NK cell activating receptors and add to the strength of signal initiated by loss of inhibition. These combined signals ultimately control NK cell cytotoxicity against cells under stress and infected with viruses.

### CAR-NK Cell Therapy

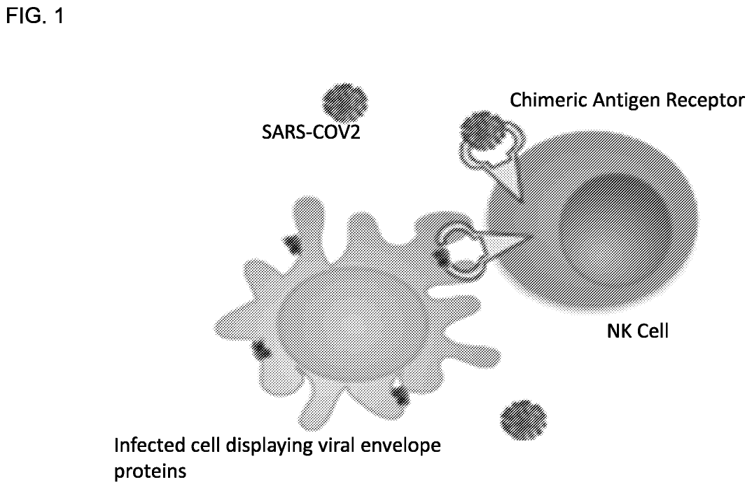

Engineering NK cells to express chimeric antigen receptors (CARs) can induce antigen-specific activation and killing. (FIG. 1) NK cells do not cause GvHD (graft versus host disease) and do not require HLA (human leukocyte antigen) matching when donor cells are infused. Thus, treatment with allogeneic CAR-NK cells has been shown to be safe in human clinical trial. Severe toxicities associated with CAR-T cell therapy are cytokine release syndrome (CRS) and immune effector cell associated neurotoxicity syndrome (ICANS). These have not been seen in the clinical use of CAR-NK cells.

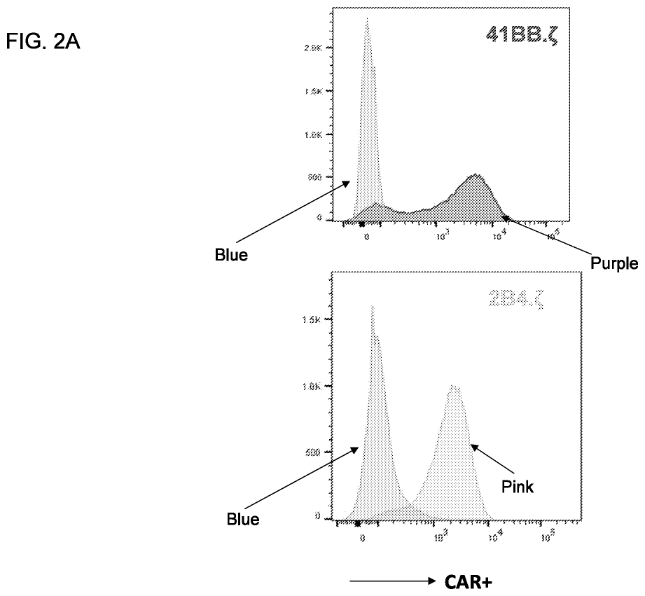

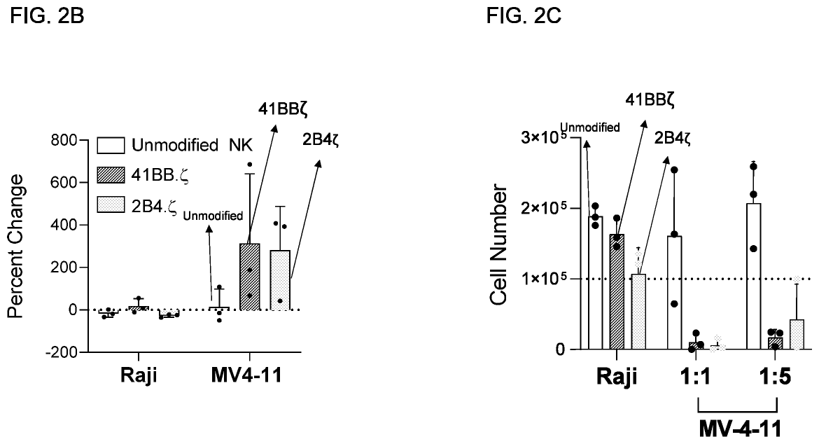

As provided herein, cell engineering is used to design several CARs that augment NK-cell activation. In preclinical evaluation, two CAR structures were observed to be superior for specific activation and cytotoxicity (FIGS. 2A-2C).

Exemplary amino acid sequences of the CARs are shown below: Underlined: Leader sequence; Bold: H84T BanLec, Italic: hinge-transmembrane, Bold and underlined: 41BB, Bold and italic: 2B4, Bold and italic and underlined: TCRζ (zeta).

H84T-BanLec.41BB.ζ
(SEQ ID NO: 6)
MDWIWRILFLVGAATGAHSNGAIKVGAWGGNGGSAFDMGPAYRIISVKI
FSGDVVDGVDVTFTYYGKTETRHYGGSGGTPHEIVLQEGEYLVGMAGEV
ANYTGAVVLGKLGFSTNKKAYGPFGNTGGTPFSLPIAAGISGFFGRGGK
FLDAIGVYLEPGGGGSTTTPAPRPPTPAPTIASQPLSLRPEACRPAAGG
AVHTRGLDFACDIYIWAPAGTCGVLLLSLVITLYCKRGRKKLLYIFKQP
FMRPVQTTQEEDGCSCRFPEEEEGGCELRVKFSRSADAPAYQQGQNQLY
NELNLGRREEYDVLDKRRGRDPEMGGKRKNPQEGLYNELQKDKMAEAYS
EIGMKGERRRGKGHDGLYQGLSTATKDTYDALHMQALPPR
H84T-BanLec.2B4.ζ
(SEQ ID NO: 7)
MDWIWRILFLVGAATGAHSNGAIKVGAWGGNGGSAFDMGPAYRIISVKI
FSGDVVDGVDVTFTYYGKTETRHYGGSGGTPHEIVLQEGEYLVGMAGEV
ANYTGAVVLGKLGFSTNKKAYGPFGNTGGTPFSLPIAAGISGFFGRGGK
FLDAIGVYLEPGGGGSQDCQNAHQEFRFWPFLVIIVILSALFLGTLACF
CVWRRKRKEKQSETSPKEFLTIYEDVKDLKTRRNHEQEQTFPGGGSTIY
SMIQSQSSAPTSQEPAYTLYSLIQPSRKSGSRKRNHSPSFNSTIYEVIG
KSQPKAQNPARLSRKELENFDVYSGAGRVKFSRSADAPAYQQGQNQLYN
ELNLGRREEYDVLDKRRGRDPEMGGKPQRRKNPQEGLYNELQKDKMAEA
YSEIGMKGERRRGKGHDGLYQGLSTATKDTYDALHMQALPPR

In examples, the leader sequence (alternatively “signal peptide”) depicted in the CAR peptides above is an N-terminal portion of the protein that assists it across the membrane of the rough endoplasmic reticulum, where it is synthesized, but which is cleaved from the protein even before the synthesis of the protein is complete, thereby producing a mature peptide (e.g., without the leader sequence).

Exemplary nucleic acid sequences and amino acid sequences of the CARs are shown below as SEQ ID NO:8 and SEQ ID NO:9 respectively:

2B4.z CAR (SEQ ID NO: 8):
ATGGACTGGATCTGGCGCATCCTGTTTCTTGTGGGAGCCGCCACAGGCG
CCCATAGCAATGGCGCTATCAAAGTTGGAGCCTGGGGCGGCAATGGCGG
CAGCGCTTTTGATATGGGCCCTGCCTACCGGATCATCAGCGTGAAGATC
TTTAGCGGCGACGTGGTGGATGGCGTGGACGTGACCTTTACCTACTACG
GCAAGACCGAGACACGGCACTATGGCGGAAGCGGAGGAACACCTCACGA
GATCGTTCTGCAAGAGGGCGAGTACCTCGTTGGAATGGCTGGCGAGGTG
GCCAACTATACAGGTGCTGTGGTGCTGGGCAAGCTGGGCTTCAGCACCA
ACAAGAAGGCCTACGGACCCTTCGGCAATACCGGCGGCACACCTTTTAG
CCTGCCTATTGCCGCCGGAATCAGCGGCTTTTTTGGCAGAGGCGGCAAG
TTCCTGGATGCCATCGGAGTGTATCTGGAACCCGGAGGGGGCGGATCCC
AGGATTGCCAGAATGCCCACCAAGAGTTCCGGTTCTGGCCCTTCCTGGT
CATCATCGTGATCCTGAGCGCCCTGTTCCTGGGCACCCTGGCCTGTTTT
TGCGTGTGGCGCAGAAAGCGCAAAGAGAAGCAGAGCGAGACAAGCCCCA
AAGAGTTCCTGACCATCTACGAGGACGTGAAGGACCTGAAAACCCGGCG
GAACCACGAGCAAGAGCAGACCTTTCCTGGCGGCGGAAGCACCATCTAC
AGCATGATCCAGAGCCAGAGCAGCGCCCCTACAAGCCAAGAGCCTGCCT
ACACACTGTACTCCCTGATCCAGCCTAGCAGAAAGAGCGGCAGCCGGAA
GAGAAATCACAGCCCCAGCTTCAACAGCACGATCTACGAAGTGATCGGC
AAGAGCCAGCCAAAGGCTCAGAACCCTGCCAGACTGAGCCGGAAAGAGC
TGGAAAACTTCGACGTGTACTCTGGGGCCGGCAGAGTGAAGTTCAGCAG
ATCAGCCGATGCTCCCGCCTATCAGCAGGGCCAGAACCAGCTGTACAAC
GAGCTGAACCTGGGGAGAAGAGAAGAGTACGACGTGCTGGACAAGCGGA
GAGGCAGAGATCCTGAGATGGGCGGAAAGCCCCAGCGGAGAAAGAATCC
TCAAGAGGGCCTGTATAATGAGCTGCAGAAAGACAAGATGGCCGAGGCC
TACAGCGAGATCGGAATGAAGGGCGAGCGCAGAAGAGGCAAGGGACACG
ATGGACTGTACCAGGGCCTGAGCACCGCCACCAAGGATACCTATGATGC
CCTGCACATGCAGGCCCTGCCTCCAAGATGA
SEQ ID NO. 9
MDWIWRILFLVGAATGAHSNGAIKVGAWGGNGGSAFDMGPAYRIISVKI
FSGDVVDGVDVTFTYYGKTETRHYGGSGGTPHEIVLQEGEYLVGMAGEV
ANYTGAVVLGKLGFSTNKKAYGPFGNTGGTPFSLPIAAGISGFFGRGGK
FLDAIGVYLEPGGGGSQDCQNAHQEFRFWPFLVIIVILSALFLGTLACF
CVWRRKRKEKQSETSPKEFLTIYEDVKDLKTRRNHEQEQTFPGGGSTIY
SMIQSQSSAPTSQEPAYTLYSLIQPSRKSGSRKRNHSPSFNSTIYEVIG
KSQPKAQNPARLSRKELENFDVYSGAGRVKFSRSADAPAYQQGQNQLYN
ELNLGRREEYDVLDKRRGRDPEMGGKPQRRKNPQEGLYNELQKDKMAEA
YSEIGMKGERRRGKGHDGLYQGLSTATKDTYDALHMQALPPR

Further exemplary nucleic acid sequences and amino acid sequences of the CARs are shown below as SEQ ID NO: 10 and SEQ ID NO: 11 respectively:

41BB.z CAR
(SEQ ID NO: 10)
ATGGACTGGATCTGGCGCATCCTGTTTCTTGTGGGAGCCGCCACAGGCG
CCCATAGCAATGGCGCTATCAAAGTTGGAGCCTGGGGCGGCAATGGCGG
CAGCGCTTTTGATATGGGCCCTGCCTACCGGATCATCAGCGTGAAGATC
TTTAGCGGCGACGTGGTGGATGGCGTGGACGTGACCTTTACCTACTACG
GCAAGACCGAGACACGGCACTATGGCGGAAGCGGAGGAACACCTCACGA
GATCGTTCTGCAAGAGGGCGAGTACCTCGTTGGAATGGCTGGCGAGGTG
GCCAACTATACAGGTGCTGTGGTGCTGGGCAAGCTGGGCTTCAGCACCA
ACAAGAAGGCCTACGGACCCTTCGGCAATACCGGCGGCACACCTTTTAG
CCTGCCTATTGCCGCCGGAATCAGCGGCTTTTTTGGCAGAGGCGGCAAG
TTCCTGGATGCCATCGGAGTGTATCTGGAACCCGGAGGGGGCGGATCCA
CAACAACCCCTGCCCCCAGACCTCCTACCCCAGCCCCTACAATTGCCAG
CCAGCCTCTGAGCCTGAGGCCCGAGGCTTGTAGACCTGCTGCTGGCGGA
GCCGTGCACACCAGAGGACTGGATTTCGCCTGCGACATCTACATCTGGG
CCCCTCTGGCCGGCACATGCGGAGTGCTGCTGCTGAGCCTCGTGATCAC
CCTGTACTGCAAGCGGGGCAGAAAGAAGCTGCTGTACATCTTCAAGCAG
CCCTTCATGCGGCCCGTGCAGACCACACAGGAAGAGGACGGCTGCTCCT
GCCGGTTCCCCGAGGAAGAAGAAGGCGGCTGCGAGCTGAGAGTGAAGTT
CTCTAGAAGCGCCGACGCCCCTGCCTACCAGCAGGGACAGAACCAGCTG
TACAACGAGCTGAACCTGGGCAGACGGGAAGAGTACGACGTGCTGGACA
AGCGGAGAGGCCGGGACCCTGAGATGGGAGGCAAGAGAAAGAACCCCCA
GGAAGGCCTGTATAACGAACTGCAGAAAGACAAGATGGCCGAGGCCTAC
AGCGAGATCGGAATGAAGGGCGAGCGGAGAAGAGGCAAGGGCCACGATG
GACTGTATCAGGGCCTGAGCACCGCCACCAAGGACACCTATGACGCCCT
GCACATGCAGGCCCTGCCCCCCAGA
SEQ ID NO. 11
MDWIWRILFLVGAATGAHSNGAIKVGAWGGNGGSAFDMGPAYRIISVKI
FSGDVVDGVDVTFTYYGKTETRHYGGSGGTPHEIVLQEGEYLVGMAGEV
ANYTGAVVLGKLGFSTNKKAYGPFGNTGGTPFSLPIAAGISGFFGRGGK
FLDAIGVYLEPGGGGSTTTPAPRPPTPAPTIASQPLSLRPEACRPAAGG
AVHTRGLDFACDIYIWAPLAGTCGVLLLSLVITLYCKRGRKKLLYIFKQ
PFMRPVQTTQEEDGCSCRFPEEEEGGCELRVKFSRSADAPAYQQGQNQL
YNELNLGRREEYDVLDKRRGRDPEMGGKRKNPQEGLYNELQKDKMAEAY
SEIGMKGERRRGKGHDGLYQGLSTATKDTYDALHMQALPPR

### CAR-NK Therapy for SARS-CoV-2 Eradication

The novel coronavirus SARS-CoV-2 has created havoc and placed international healthcare systems under unprecedented stress. Lack of prior exposure and a high fatality rate have together resulted in a global pandemic. Currently, there is no treatment proven effective against SARS-CoV-2. Critically ill patients demonstrate immune dysregulation, with both reduced number and function of effector cells. NK cell dysfunction impacts host ability to clear viral infections. Adoptive transfer of healthy donor immune cells can temporarily repopulate host innate immunity. NK cells can be frozen and stored as an “off-the-shelf” product, readily available when needed. However, unmodified NK cells may demonstrate non-specific activation and uncontrolled activity. Engineering NK cells with Chimeric Antigen Receptors (CARs) can direct antiviral activity and provision the cells with boosted killing potential.

CAR functionality and safety is heavily dependent on target selection. The SARS-CoV-2 viral envelope is heavily glycosylated, with expression of glycosites distinct from those detected on healthy human cells. CAR targeting of T cells to membranous high mannose containing glycoproteins was shown. NK cells expressing a CAR comprised of a targeting domain that binds high mannose binds to SARS-CoV-2 envelope proteins. Indeed, the spike(S) protein, critical for viral cell entry via binding of the human ACE2 receptor, displays viral-specific glycosites.

BanLec is a lectin derived from bananas that when modified with a single amino acid point mutation (H84T) specifically binds high mannose, without triggering T cell mitogenicity. The resultant H84T-BanLec can bind glycoproteins incorporated into the envelopes of multiple viruses. H84T-BanLec alone has been shown to reduce viral infectivity and promote survival in animal models of viral infection. However, lectins are subject to chemical and biomolecular degradation, making administration and pharmacologic stability a barrier to human treatment.

H84T-BanLec was engineered as the targeting moiety of a CAR expressed on T cells. Due to rapid cell division and dysregulated post-translational processing, transformed cells can display abnormally glycosylated membrane proteins. Indeed, H84T-BanLec.CAR T cells can promote specific killing of lung cancer cells, without affecting normal lung fibroblasts. Though powerful cytotoxic immune effectors, T cells are not an ideal cell therapy agent to treat SARS-CoV-2 infection.

As provided herein, the safety profile and favorable characteristics of NK cells is shown. Specificity is improved and activation is boosted by expressing H84T-BanLec.CAR on NK cells. H84T-BanLec. CAR NK cell binding and activation by SARS-CoV-2 envelope proteins is shown as a step to further clinical development.

### Viral Glycoprotein

A viral envelope is the outermost layer of many types of viruses. It protects the genetic material in their life-cycle when traveling between host cells. Not all viruses have envelopes.

The envelopes are typically derived from portions of the host cell membranes (phospholipids and proteins), but include some viral glycoproteins. They may help viruses avoid the host immune system. Glycoproteins on the surface of the envelope serve to identify and bind to receptor on the host's membrane. The viral envelope then fuses with the host's membrane, allowing the capsid and viral genome to enter and infect the host. All enveloped viruses also have a capsid, another protein layer, between the envelope and the genome.

### Spike Proteins

The S protein is a highly glycosylated and large type I transmembrane fusion protein that is made up of 1,160 to 1,400 amino acids, depending upon the type of virus. As compared to the M and E proteins that are primarily involved in virus assembly, the S protein plays a crucial role in binding host cells and initiating infection.

S proteins of coronaviruses can be divided into two important functional subunits, of which include the N-terminal S1 subunit, which forms of the globular head of the S protein, and the C-terminal S2 region that forms the stalk of the protein and is directly embedded into the viral envelope. Upon interaction with a potential host cell, the S1 subunit will recognize and bind to receptors on the host cell, whereas the S2 subunit, which is the most conserved component of the S protein, will be responsible for fusing the envelope of the virus with the host cell membrane.

Coronavirus infect human respiratory epithelial cells through interaction with the human ACE2 (angiotensin-converting enzyme 2) receptor. The spike protein is a large type I transmembrane protein containing two subunits, S1 and S2. S1 mainly contains a receptor binding domain (RBD), which is responsible for recognizing the ACE2 cell surface receptor. S2 contains basic elements needed for the membrane fusion. The S protein plays key parts in the induction of neutralizing-antibody and T-cell responses, as well as protective immunity.

An amino acid sequence of a surface glycoprotein for SARS-CoV-2 (severe acute respiratory syndrome coronavirus 2) is publically available at the NCBI database under accession number: QHD43416.1, incorporated herein by reference (SEQ ID NO: 12).

| | |
|---|---|
| 1 | mfvflvllpl vssqcvnltt rtqlppaytn sftrgvyypd kvfrssvlhs tqdlflpffs |
| 61 | nvtwfhaihv sgtngtkrfd npvlpfndgv yfasteksni irgwifgttl dsktqslliv |
| 121 | nnatnvvikv cefqfondpf lgvyyhknnk swmesefrvy ssannctfey vsqpflmdle |
| 181 | gkqgnfknlr efvfknidgy fkiyskhtpi nlvrdlpqgf saleplvdlp iginitrfqt |
| 241 | llalhrsylt pgdsssgwta gaaayyvgyl qprtfllkyn engtitdavd caldplsetk |
| 301 | ctlksftvek giyqtsnfrv qptesivrfp nitnlcpfge vfnatrfasv yawnrkrisn |
| 361 | cvadysvlyn sasfstfkcy gvsptkindl cftnvyadsf virgdevrqi apgqtgkiad |
| 421 | ynyklpddft gcviawnsnn ldskvggnyn ylyrlfrksn lkpferdist eiyqagstpc |
| 481 | ngvegfncyf plqsygfqpt ngvgyqpyrv vvlsfellha patvcgpkks tnlvknkcvn |
| 541 | fnfngltgtg vltesnkkfl pfqqfgrdia dttdavrdpq tleilditpc sfggvsvitp |
| 601 | gtntsnqvav lyqdvnctev pvaihadqlt ptwrvystgs nvfqtragcl igaehvnnsy |
| 661 | ecdipigagi casyqtqtns prrarsvasq siiaytmslg aensvaysnn siaiptnfti |
| 721 | svtteilpvs mtktsvdctm yicgdstecs nlllqygsfc tqlnraltgi aveqdkntqe |
| 781 | vfaqvkqiyk tppikdfggf nfsqilpdps kpskrsfied llfnkvtlad agfikqygdc |
| 841 | lgdiaardli caqkfngltv lpplltdemi aqytsallag titsgwtfga gaalqipfam |
| 901 | qmayrfngig vtqnvlyenq klianqfnsa igkiqdslss tasalgklqd vvnqnaqaln |
| 961 | tlvkqlssnf gaissvlndi lsrldkveae vqidrlitgr lqslqtyvtq qliraaeira |
| 1021 | sanlaatkms ecvlgqskrv dfcgkgyhlm sfpqsaphgv vflhvtyvpa qeknfttapa |
| 1081 | ichdgkahfp regvfvsngt hwfvtqrnfy epqiittdnt fvsgncdvvi givnntvydp |
| 1141 | lqpeldsfke eldkyfknht spdvdlgdis ginasvvniq keidrlneva knlneslidl |
| 1201 | qelgkyegyi kwpwyiwlgf iagliaivmv timlccmtsc csclkgccsc gscckfdedd |
| 1261 | sepvlkgvkl hyt |

A nucleotide sequence of a surface glycoprotein for SARS-COV2 (severe acute respiratory syndrome coronavirus 2) is publically available at the NCBI database under accession number: MN908947.31, incorporated herein by reference (SEQ ID NO: 13). The start and stop codons are bold and underlined.

| | |
|---|---|
| 1 | attaaaggtt tataccttcc caggtaacaa accaaccaac tttcgatctc ttgtagatct |
| 61 | gttctctaaa cgaactttaa aatctgtgtg gctgtcactc ggctgcatgc ttagtgcact |
| 121 | cacgcagtat aattaataac taattactgt cgttgacagg acacgagtaa ctcgtctatc |
| 181 | ttctgcaggc tgcttacggt ttcgtccgtg ttgcagccga tcatcagcac atctaggttt |
| 241 | cgtccgggtg tgaccgaaag gtaagatgga gagccttgtc cctggtttca acgagaaaac |
| 301 | acacgtccaa ctcagtttgc ctgttttaca ggttcgcgac gtgctcgtac gtggctttgg |
| 361 | agactccgtg gaggaggtct tatcagaggc acgtcaacat cttaaagatg gcacttgtgg |
| 421 | cttagtagaa gttgaaaaag gcgttttgcc tcaacttgaa cagccctatg tgttcatcaa |
| 481 | acgttcggat gctcgaactg cacctcatgg tcatgttatg gttgagctgg tagcagaact |
| 541 | cgaaggcatt cagtacggtc gtagtggtga gacacttggt gtccttgtcc ctcatgtggg |
| 601 | cgaaatacca gtggcttacc gcaaggttct tcttcgtaag aacggtaata aaggagctgg |
| 661 | tggccatagt tacggcgccg atctaaagtc atttgactta ggcgacgagc ttggcactga |
| 721 | tccttatgaa gattttcaag aaaactggaa cactaaacat agcagtggtg ttacccgtga |
| 781 | actcatgcgt gagcttaacg gaggggcata cactcgctat gtcgataaca acttctgtgg |
| 841 | ccctgatggc taccctcttg agtgcattaa agaccttcta gcacgtgctg gtaaagcttc |
| 901 | atgcactttg tccgaacaac tggactttat tgacactaag aggggtgtat actgctgccg |
| 961 | tgaacatgag catgaaattg cttggtacac ggaacgttct gaaaagagct atgaattgca |
| 1021 | gacacctttt gaaattaaat tggcaaagaa atttgacacc ttcaatgggg aatgtccaaa |
| 1081 | ttttgtattt cccttaaatt ccataatcaa gactattcaa ccaagggttg aaaagaaaaa |
| 1141 | gcttgatggc tttatgggta gaattcgatc tgtctatcca gttgcgtcac caaatgaatg |
| 1201 | caaccaaatg tgcctttcaa ctctcatgaa gtgtgatcat tgtggtgaaa cttcatggca |
| 1261 | gacgggcgat tttgttaaag ccacttgcga attttgtggc actgagaatt tgactaaaga |
| 1321 | aggtgccact acttgtggtt acttacccca aaatgctgtt gttaaaattt attgtccagc |
| 1381 | atgtcacaat tcagaagtag gacctgagca tagtcttgcc gaataccata atgaatctgg |
| 1441 | cttgaaaacc attcttcgta agggtggtcg cactattgcc tttggaggct gtgtgttctc |
| 1501 | ttatgttggt tgccataaca agtgtgccta ttgggttcca cgtgctagcg ctaacatagg |
| 1561 | ttgtaaccat acaggtgttg ttggagaagg ttccgaaggt cttaatgaca accttcttga |
| 1621 | aatactccaa aaagagaaag tcaacatcaa tattgttggt gactttaaac ttaatgaaga |
| 1681 | gatcgccatt attttggcat ctttttctgc ttccacaagt gcttttgtgg aaactgtgaa |
| 1741 | aggtttggat tataaagcat tcaaacaaat tgttgaatcc tgtggtaatt ttaaagttac |
| 1801 | aaaaggaaaa gctaaaaaag gtgcctggaa tattggtgaa cagaaatcaa tactgagtcc |
| 1861 | tctttatgca tttgcatcag aggctgctcg tgttgtacga tcaattttct cccgcactct |
| 1921 | tgaaactgct caaaattctg tgcgtgtttt acagaaggcc gctataacaa tactagatgg |
| 1981 | aatttcacag tattcactga gactcattga tgctatgatg ttcacatctg atttggctac |
| 2041 | taacaatcta gttgtaatgg cctacattac aggtggtgtt gttcagttga cttcgcagtg |
| 2101 | gctaactaac atctttggca ctgtttatga aaaactcaaa cccgtccttg attggcttga |
| 2161 | agagaagttt aaggaaggtg tagagtttct tagagacggt tgggaaattg ttaaatttat |
| 2221 | ctcaacctgt gcttgtgaaa ttgtcggtgg acaaattgtc acctgtgcaa aggaaattaa |
| 2281 | ggagagtgtt cagacattct ttaagcttgt aaataaattt ttggctttgt gtgctgactc |
| 2341 | tatcattatt ggtggagcta aacttaaagc cttgaattta ggtgaaacat ttgtcacgca |
| 2401 | ctcaaaggga ttgtacagaa agtgtgttaa atccagagaa gaaactggcc tactcatgcc |
| 2461 | tctaaaagcc ccaaaagaaa ttatcttctt agagggagaa acacttccca cagaagtgtt |
| 2521 | aacagaggaa gttgtcttga aaactggtga tttacaacca ttagaacaac ctactagtga |
| 2581 | agctgttgaa gctccattgg ttggtacacc agtttgtatt aacgggctta tgttgctcga |
| 2641 | aatcaaagac acagaaaagt actgtgccct tgcacctaat atgatggtaa caaacaatac |
| 2701 | cttcacactc aaaggcggtg caccaacaaa ggttactttt ggtgatgaca ctgtgataga |
| 2761 | agtgcaaggt tacaagagtg tgaatatcac ttttgaactt gatgaaagga ttgataaagt |
| 2821 | acttaatgag aagtgctctg cctatacagt tgaactcggt acagaagtaa atgagttcgc |
| 2881 | ctgtgttgtg gcagatgctg tcataaaaac tttgcaacca gtatctgaat tacttacacc |
| 2941 | actgggcatt gatttagatg agtggagtat ggctacatac tacttatttg atgagtctgg |
| 3001 | tgagtttaaa ttggcttcac atatgtattg ttctttctac cctccagatg aggatgaaga |
| 3061 | agaaggtgat tgtgaagaag aagagtttga gccatcaact caatatgagt atggtactga |
| 3121 | agatgattac caaggtaaac ctttggaatt tggtgccact tctgctgctc ttcaacctga |
| 3181 | agaagagcaa gaagaagatt ggttagatga tgatagtcaa caaactgttg gtcaacaaga |
| 3241 | cggcagtgag gacaatcaga caactactat tcaaacaatt gttgaggttc aacctcaatt |
| 3301 | agagatggaa cttacaccag ttgttcagac tattgaagtg aatagtttta gtggttattt |
| 3361 | aaaacttact gacaatgtat acattaaaaa tgcagacatt gtggaagaag ctaaaaaggt |
| 3421 | aaaaccaaca gtggttgtta atgcagccaa tgtttacctt aaacatggag gaggtgttgc |
| 3481 | aggagcctta aataaggcta ctaacaatgc catgcaagtt gaatctgatg attacatagc |
| 3541 | tactaatgga ccacttaaag tgggtggtag ttgtgtttta agcggacaca atcttgctaa |
| 3601 | acactgtctt catgttgtcg gcccaaatgt taacaaaggt gaagacattc aacttcttaa |
| 3661 | gagtgcttat gaaaatttta atcagcacga agttctactt gcaccattat tatcagctgg |
| 3721 | tatttttggt gctgacccta tacattcttt aagagtttgt gtagatactg ttcgcacaaa |
| 3781 | tgtctactta gctgtctttg ataaaaatct ctatgacaaa cttgtttcaa gctttttgga |
| 3841 | aatgaagagt gaaaagcaag ttgaacaaaa gatcgctgag attcctaaag aggaagttaa |
| 3901 | gccatttata actgaaagta aaccttcagt tgaacagaga aaacaagatg ataagaaaat |
| 3961 | caaagcttgt gttgaagaag ttacaacaac tctggaagaa actaagttcc tcacagaaaa |
| 4021 | cttgttactt tatattgaca ttaatggcaa tcttcatcca gattctgcca ctcttgttag |
| 4081 | tgacattgac atcactttct taaagaaaga tgctccatat atagtgggtg atgttgttca |
| 4141 | agagggtgtt ttaactgctg tggttatacc tactaaaaag gctggtggca ctactgaaat |
| 4201 | gctagcgaaa gctttgagaa aagtgccaac agacaattat ataaccactt acccgggtca |
| 4261 | gggtttaaat ggttacactg tagaggaggc aaagacagtg cttaaaaagt gtaaaagtgc |
| 4321 | cttttacatt ctaccatcta ttatctctaa tgagaagcaa gaaattcttg gaactgtttc |
| 4381 | ttggaatttg cgagaaatgc ttgcacatgc agaagaaaca cgcaaattaa tgcctgtctg |
| 4441 | tgtggaaact aaagccatag tttcaactat acagcgtaaa tataagggta ttaaaataca |
| 4501 | agagggtgtg gttgattatg gtgctagatt ttacttttac accagtaaaa caactgtagc |
| 4561 | gtcacttatc aacacactta acgatctaaa tgaaactctt gttacaatgc cacttggcta |
| 4621 | tgtaacacat ggcttaaatt tggaagaagc tgctcggtat atgagatctc tcaaagtgcc |
| 4681 | agctacagtt tctgtttctt cacctgatgc tgttacagcg tataatggtt atcttacttc |
| 4741 | ttcttctaaa acacctgaag aacattttat tgaaaccatc tcacttgctg gttcctataa |
| 4801 | agattggtcc tattctggac aatctacaca actaggtata gaatttctta agagaggtga |
| 4861 | taaaagtgta tattacacta gtaatcctac cacattccac ctagatggtg aagttatcac |
| 4921 | ctttgacaat cttaagacac ttctttcttt gagagaagtg aggactatta aggtgtttac |
| 4981 | aacagtagac aacattaacc tccacacgca agttgtggac atgtcaatga catatggaca |
| 5041 | acagtttggt ccaacttatt tggatggagc tgatgttact aaaataaaac ctcataattc |
| 5101 | acatgaaggt aaaacatttt atgttttacc taatgatgac actctacgtg ttgaggcttt |
| 5161 | tgagtactac cacacaactg atcctagttt tctgggtagg tacatgtcag cattaaatca |
| 5221 | cactaaaaag tggaaatacc cacaagttaa tggtttaact tctattaaat gggcagataa |
| 5281 | caactgttat cttgccactg cattgttaac actccaacaa atagagttga agtttaatcc |
| 5341 | acctgctcta caagatgctt attacagagc aagggctggt gaagctgcta acttttgtgc |
| 5401 | acttatctta gcctactgta ataagacagt aggtgagtta ggtgatgtta gagaaacaat |
| 5461 | gagttacttg tttcaacatg ccaatttaga ttcttgcaaa agagtcttga acgtggtgtg |
| 5521 | taaaacttgt ggacaacagc agacaaccct taagggtgta gaagctgtta tgtacatggg |
| 5581 | cacactttct tatgaacaat ttaagaaagg tgttcagata ccttgtacgt gtggtaaaca |
| 5641 | agctacaaaa tatctagtac aacaggagtc accttttgtt atgatgtcag caccacctgc |
| 5701 | tcagtatgaa cttaagcatg gtacatttac ttgtgctagt gagtacactg gtaattacca |
| 5761 | gtgtggtcac tataaacata taacttctaa agaaactttg tattgcatag acggtgcttt |
| 5821 | acttacaaag tcctcagaat acaaaggtcc tattacggat gttttctaca aagaaaacag |
| 5881 | ttacacaaca accataaaac cagttactta taaattggat ggtgttgttt gtacagaaat |
| 5941 | tgaccctaag ttggacaatt attataagaa agacaattct tatttcacag agcaaccaat |
| 6001 | tgatcttgta ccaaaccaac catatccaaa cgcaagcttc gataatttta agtttgtatg |
| 6061 | tgataatatc aaatttgctg atgatttaaa ccagttaact ggttataaga aacctgcttc |
| 6121 | aagagagctt aaagttacat ttttccctga cttaaatggt gatgtggtgg ctattgatta |
| 6181 | taaacactac acaccctctt ttaagaaagg agctaaattg ttacataaac ctattgtttg |
| 6241 | gcatgttaac aatgcaacta ataaagccac gtataaacca aatacctggt gtatacgttg |
| 6301 | tctttggagc acaaaaccag ttgaaacatc aaattcgttt gatgtactga agtcagagga |
| 6361 | cgcgcaggga atggataatc ttgcctgcga agatctaaaa ccagtctctg aagaagtagt |
| 6421 | ggaaaatcct accatacaga aagacgttct tgagtgtaat gtgaaaacta ccgaagttgt |
| 6481 | aggagacatt atacttaaac cagcaaataa tagtttaaaa attacagaag aggttggcca |
| 6541 | cacagatcta atggctgctt atgtagacaa ttctagtctt actattaaga aacctaatga |
| 6601 | attatctaga gtattaggtt tgaaaaccct tgctactcat ggtttagctg ctgttaatag |
| 6661 | tgtcccttgg gatactatag ctaattatgc taagcctttt cttaacaaag ttgttagtac |
| 6721 | aactactaac atagttacac ggtgtttaaa ccgtgtttgt actaattata tgccttattt |
| 6781 | ctttacttta ttgctacaat tgtgtacttt tactagaagt acaaattcta gaattaaagc |
| 6841 | atctatgccg actactatag caaagaatac tgttaagagt gtcggtaaat tttgtctaga |
| 6901 | ggcttcattt aattatttga agtcacctaa tttttctaaa ctgataaata ttataatttg |
| 6961 | gtttttacta ttaagtgttt gcctaggttc tttaatctac tcaaccgctg ctttaggtgt |
| 7021 | tttaatgtct aatttaggca tgccttctta ctgtactggt tacagagaag gctatttgaa |
| 7081 | ctctactaat gtcactattg caacctactg tactggttct ataccttgta gtgtttgtct |
| 7141 | tagtggttta gattctttag acacctatcc ttctttagaa actatacaaa ttaccatttc |
| 7201 | atcttttaaa tgggatttaa ctgcttttgg cttagttgca gagtggtttt tggcatatat |
| 7261 | tcttttcact aggtttttct atgtacttgg attggctgca atcatgcaat tgtttttcag |
| 7321 | ctattttgca gtacatttta ttagtaattc ttggcttatg tggttaataa ttaatcttgt |
| 7381 | acaaatggcc ccgatttcag ctatggttag aatgtacatc ttctttgcat cattttatta |
| 7441 | tgtatggaaa agttatgtgc atgttgtaga cggttgtaat tcatcaactt gtatgatgtg |
| 7501 | ttacaaacgt aatagagcaa caagagtcga atgtacaact attgttaatg gtgttagaag |
| 7561 | gtccttttat gtctatgcta atggaggtaa aggcttttgc aaactacaca attggaattg |
| 7621 | tgttaattgt gatacattct gtgctggtag tacatttatt agtgatgaag ttgcgagaga |
| 7681 | cttgtcacta cagtttaaaa gaccaataaa tcctactgac cagtcttctt acatcgttga |
| 7741 | tagtgttaca gtgaagaatg gttccatcca tctttacttt gataaagctg gtcaaaagac |
| 7801 | ttatgaaaga cattctctct ctcattttgt taacttagac aacctgagag ctaataacac |
| 7861 | taaaggttca ttgcctatta atgttatagt ttttgatggt aaatcaaaat gtgaagaatc |
| 7921 | atctgcaaaa tcagcgtctg tttactacag tcagcttatg tgtcaaccta tactgttact |
| 7981 | agatcaggca ttagtgtctg atgttggtga tagtgcggaa gttgcagtta aaatgtttga |
| 8041 | tgcttacgtt aatacgtttt catcaacttt taacgtacca atggaaaaac tcaaaacact |
| 8101 | agttgcaact gcagaagctg aacttgcaaa gaatgtgtcc ttagacaatg tcttatctac |
| 8161 | ttttatttca gcagctcggc aagggtttgt tgattcagat gtagaaacta aagatgttgt |
| 8221 | tgaatgtctt aaattgtcac atcaatctga catagaagtt actggcgata gttgtaataa |
| 8281 | ctatatgctc acctataaca aagttgaaaa catgacaccc cgtgaccttg gtgcttgtat |
| 8341 | tgactgtagt gcgcgtcata ttaatgcgca ggtagcaaaa agtcacaaca ttgctttgat |
| 8401 | atggaacgtt aaagatttca tgtcattgtc tgaacaacta cgaaaacaaa tacgtagtgc |
| 8461 | tgctaaaaag aataacttac cttttaagtt gacatgtgca actactagac aagttgttaa |
| 8521 | tgttgtaaca acaaagatag cacttaaggg tggtaaaatt gttaataatt ggttgaagca |
| 8581 | gttaattaaa gttacacttg tgttcctttt tgttgctgct attttctatt taataacacc |
| 8641 | tgttcatgtc atgtctaaac atactgactt ttcaagtgaa atcataggat acaaggctat |
| 8701 | tgatggtggt gtcactcgtg acatagcatc tacagatact tgttttgcta acaaacatgc |
| 8761 | tgattttgac acatggttta gccagcgtgg tggtagttat actaatgaca aagcttgccc |
| 8821 | attgattgct gcagtcataa caagagaagt gggttttgtc gtgcctggtt tgcctggcac |
| 8881 | gatattacgc acaactaatg gtgacttttt gcatttctta cctagagttt ttagtgcagt |
| 8941 | tggtaacatc tgttacacac catcaaaact tatagagtac actgactttg caacatcagc |
| 9001 | ttgtgttttg gctgctgaat gtacaatttt taaagatgct tctggtaagc cagtaccata |
| 9061 | ttgttatgat accaatgtac tagaaggttc tgttgcttat gaaagtttac gccctgacac |
| 9121 | acgttatgtg ctcatggatg gctctattat tcaatttcct aacacctacc ttgaaggttc |
| 9181 | tgttagagtg gtaacaactt ttgattctga gtactgtagg cacggcactt gtgaaagatc |
| 9241 | agaagctggt gtttgtgtat ctactagtgg tagatgggta cttaacaatg attattacag |
| 9301 | atctttacca ggagttttct gtggtgtaga tgctgtaaat ttacttacta atatgtttac |
| 9361 | accactaatt caacctattg gtgctttgga catatcagca tctatagtag ctggtggtat |
| 9421 | tgtagctatc gtagtaacat gccttgccta ctattttatg aggtttagaa gagcttttgg |
| 9481 | tgaatacagt catgtagttg cctttaatac tttactattc cttatgtcat tcactgtact |
| 9541 | ctgtttaaca ccagtttact cattcttacc tggtgtttat tctgttattt acttgtactt |
| 9601 | gacattttat cttactaatg atgtttcttt tttagcacat attcagtgga tggttatgtt |
| 9661 | cacaccttta gtacctttct ggataacaat tgcttatatc atttgtattt ccacaaagca |
| 9721 | tttctattgg ttctttagta attacctaaa gagacgtgta gtctttaatg gtgtttcctt |
| 9781 | tagtactttt gaagaagctg cgctgtgcac ctttttgtta aataaagaaa tgtatctaaa |
| 9841 | gttgcgtagt gatgtgctat tacctcttac gcaatataat agatacttag ctctttataa |
| 9901 | taagtacaag tattttagtg gagcaatgga tacaactagc tacagagaag ctgcttgttg |
| 9961 | tcatctcgca aaggctctca atgacttcag taactcaggt tctgatgttc tttaccaacc |
| 10021 | accacaaacc tctatcacct cagctgtttt gcagagtggt tttagaaaaa tggcattccc |
| 10081 | atctggtaaa gttgagggtt gtatggtaca agtaacttgt ggtacaacta cacttaacgg |
| 10141 | tctttggctt gatgacgtag tttactgtcc aagacatgtg atctgcacct ctgaagacat |
| 10201 | gcttaaccct aattatgaag atttactcat tcgtaagtct aatcataatt tcttggtaca |
| 10261 | ggctggtaat gttcaactca gggttattgg acattctatg caaaattgtg tacttaagct |
| 10321 | taaggttgat acagccaatc ctaagacacc taagtataag tttgttcgca ttcaaccagg |
| 10381 | acagactttt tcagtgttag cttgttacaa tggttcacca tctggtgttt accaatgtgc |
| 10441 | tatgaggccc aatttcacta ttaagggttc attccttaat ggttcatgtg gtagtgttgg |
| 10501 | ttttaacata gattatgact gtgtctcttt ttgttacatg caccatatgg aattaccaac |
| 10561 | tggagttcat gctggcacag acttagaagg taacttttat ggaccttttg ttgacaggca |
| 10621 | aacagcacaa gcagctggta cggacacaac tattacagtt aatgttttag cttggttgta |
| 10681 | cgctgctgtt ataaatggag acaggtggtt tctcaatcga tttaccacaa ctcttaatga |
| 10741 | ctttaacctt gtggctatga agtacaatta tgaacctcta acacaagacc atgttgacat |
| 10801 | actaggacct ctttctgctc aaactggaat tgccgtttta gatatgtgtg cttcattaaa |
| 10861 | agaattactg caaaatggta tgaatggacg taccatattg ggtagtgctt tattagaaga |
| 10921 | tgaatttaca ccttttgatg ttgttagaca atgctcaggt gttactttcc aaagtgcagt |
| 10981 | gaaaagaaca atcaagggta cacaccactg gttgttactc acaattttga cttcactttt |
| 11041 | agttttagtc cagagtactc aatggtcttt gttctttttt ttgtatgaaa atgccttttt |
| 11101 | accttttgct atgggtatta ttgctatgtc tgcttttgca atgatgtttg tcaaacataa |
| 11161 | gcatgcattt ctctgtttgt ttttgttacc ttctcttgcc actgtagctt attttaatat |
| 11221 | ggtctatatg cctgctagtt gggtgatgcg tattatgaca tggttggata tggttgatac |
| 11281 | tagtttgtct ggttttaagc taaaagactg tgttatgtat gcatcagctg tagtgttact |
| 11341 | aatccttatg acagcaagaa ctgtgtatga tgatggtgct aggagagtgt ggacacttat |
| 11401 | gaatgtcttg acactcgttt ataaagttta ttatggtaat gctttagatc aagccatttc |
| 11461 | catgtgggct cttataatct ctgttacttc taactactca ggtgtagtta caactgtcat |
| 11521 | gtttttggcc agaggtattg tttttatgtg tgttgagtat tgccctattt tcttcataac |
| 11581 | tggtaataca cttcagtgta taatgctagt ttattgtttc ttaggctatt tttgtacttg |
| 11641 | ttactttggc ctcttttgtt tactcaaccg ctactttaga ctgactcttg gtgtttatga |
| 11701 | ttacttagtt tctacacagg agtttagata tatgaattca cagggactac tcccacccaa |
| 11761 | gaatagcata gatgccttca aactcaacat taaattgttg ggtgttggtg gcaaaccttg |
| 11821 | tatcaaagta gccactgtac agtctaaaat gtcagatgta aagtgcacat cagtagtctt |
| 11881 | actctcagtt ttgcaacaac tcagagtaga atcatcatct aaattgtggg ctcaatgtgt |
| 11941 | ccagttacac aatgacattc tcttagctaa agatactact gaagcctttg aaaaaatggt |
| 12001 | ttcactactt tctgttttgc tttccatgca gggtgctgta gacataaaca agctttgtga |
| 12061 | agaaatgctg gacaacaggg caaccttaca agctatagcc tcagagttta gttcccttcc |
| 12121 | atcatatgca gcttttgcta ctgctcaaga agcttatgag caggctgttg ctaatggtga |
| 12181 | ttctgaagtt gttcttaaaa agttgaagaa gtctttgaat gtggctaaat ctgaatttga |
| 12241 | ccgtgatgca gccatgcaac gtaagttgga aaagatggct gatcaagcta tgacccaaat |
| 12301 | gtataaacag gctagatctg aggacaagag ggcaaaagtt actagtgcta tgcagacaat |
| 12361 | gcttttcact atgcttagaa agttggataa tgatgcactc aacaacatta tcaacaatgc |
| 12421 | aagagatggt tgtgttccct tgaacataat acctcttaca acagcagcca aactaatggt |
| 12481 | tgtcatacca gactataaca catataaaaa tacgtgtgat ggtacaacat ttacttatgc |
| 12541 | atcagcattg tgggaaatcc aacaggttgt agatgcagat agtaaaattg ttcaacttag |
| 12601 | tgaaattagt atggacaatt cacctaattt agcatggcct cttattgtaa cagctttaag |
| 12661 | ggccaattct gctgtcaaat tacagaataa tgagcttagt cctgttgcac tacgacagat |
| 12721 | gtcttgtgct gccggtacta cacaaactgc ttgcactgat gacaatgcgt tagcttacta |
| 12781 | caacacaaca aagggaggta ggtttgtact tgcactgtta tccgatttac aggatttgaa |
| 12841 | atgggctaga ttccctaaga gtgatggaac tggtactatc tatacagaac tggaaccacc |
| 12901 | ttgtaggttt gttacagaca cacctaaagg tcctaaagtg aagtatttat actttattaa |
| 12961 | aggattaaac aacctaaata gaggtatggt acttggtagt ttagctgcca cagtacgtct |
| 13021 | acaagctggt aatgcaacag aagtgcctgc caattcaact gtattatctt tctgtgcttt |
| 13081 | tgctgtagat gctgctaaag cttacaaaga ttatctagct agtgggggac aaccaatcac |
| 13141 | taattgtgtt aagatgttgt gtacacacac tggtactggt caggcaataa cagttacacc |
| 13201 | ggaagccaat atggatcaag aatcctttgg tggtgcatcg tgttgtctgt actgccgttg |
| 13261 | ccacatagat catccaaatc ctaaaggatt ttgtgactta aaaggtaagt atgtacaaat |
| 13321 | acctacaact tgtgctaatg accctgtggg ttttacactt aaaaacacag tctgtaccgt |
| 13381 | ctgcggtatg tggaaaggtt atggctgtag ttgtgatcaa ctccgcgaac ccatgcttca |
| 13441 | gtcagctgat gcacaatcgt ttttaaacgg gtttgcggtg taagtgcagc ccgtcttaca |
| 13501 | ccgtgcggca caggcactag tactgatgtc gtatacaggg cttttgacat ctacaatgat |
| 13561 | aaagtagctg gttttgctaa attcctaaaa actaattgtt gtcgcttcca agaaaaggac |
| 13621 | gaagatgaca atttaattga ttcttacttt gtagttaaga gacacacttt ctctaactac |
| 13681 | caacatgaag aaacaattta taatttactt aaggattgtc cagctgttgc taaacatgac |
| 13741 | ttctttaagt ttagaataga cggtgacatg gtaccacata tatcacgtca acgtcttact |
| 13801 | aaatacacaa tggcagacct cgtctatgct ttaaggcatt ttgatgaagg taattgtgac |
| 13861 | acattaaaag aaatacttgt cacatacaat tgttgtgatg atgattattt caataaaaag |
| 13921 | gactggtatg attttgtaga aaacccagat atattacgcg tatacgccaa cttaggtgaa |
| 13981 | cgtgtacgcc aagctttgtt aaaaacagta caattctgtg atgccatgcg aaatgctggt |
| 14041 | attgttggtg tactgacatt agataatcaa gatctcaatg gtaactggta tgatttcggt |
| 14101 | gatttcatac aaaccacgcc aggtagtgga gttcctgttg tagattctta ttattcattg |
| 14161 | ttaatgccta tattaacctt gaccagggct ttaactgcag agtcacatgt tgacactgac |
| 14221 | ttaacaaagc cttacattaa gtgggatttg ttaaaatatg acttcacgga agagaggtta |
| 14281 | aaactctttg accgttattt taaatattgg gatcagacat accacccaaa ttgtgttaac |
| 14341 | tgtttggatg acagatgcat tctgcattgt gcaaacttta atgttttatt ctctacagtg |
| 14401 | ttcccaccta caagttttgg accactagtg agaaaaatat ttgttgatgg tgttccattt |
| 14461 | gtagtttcaa ctggatacca cttcagagag ctaggtgttg tacataatca ggatgtaaac |
| 14521 | ttacatagct ctagacttag ttttaaggaa ttacttgtgt atgctgctga ccctgctatg |
| 14581 | cacgctgctt ctggtaatct attactagat aaacgcacta cgtgcttttc agtagctgca |
| 14641 | cttactaaca atgttgcttt tcaaactgtc aaacccggta attttaacaa agacttctat |
| 14701 | gactttgctg tgtctaaggg tttctttaag gaaggaagtt ctgttgaatt aaaacacttc |
| 14761 | ttctttgctc aggatggtaa tgctgctatc agcgattatg actactatcg ttataatcta |
| 14821 | ccaacaatgt gtgatatcag acaactacta tttgtagttg aagttgttga taagtacttt |
| 14881 | gattgttacg atggtggctg tattaatgct aaccaagtca tcgtcaacaa cctagacaaa |
| 14941 | tcagctggtt ttccatttaa taaatggggt aaggctagac tttattatga ttcaatgagt |
| 15001 | tatgaggatc aagatgcact tttcgcatat acaaaacgta atgtcatccc tactataact |
| 15061 | caaatgaatc ttaagtatgc cattagtgca aagaatagag ctcgcaccgt agctggtgtc |
| 15121 | tctatctgta gtactatgac caatagacag tttcatcaaa aattattgaa atcaatagcc |
| 15181 | gccactagag gagctactgt agtaattgga acaagcaaat tctatggtgg ttggcacaac |
| 15241 | atgttaaaaa ctgtttatag tgatgtagaa aaccctcacc ttatgggttg ggattatcct |
| 15301 | aaatgtgata gagccatgcc taacatgctt agaattatgg cctcacttgt tcttgctcgc |
| 15361 | aaacatacaa cgtgttgtag cttgtcacac cgtttctata gattagctaa tgagtgtgct |
| 15421 | caagtattga gtgaaatggt catgtgtggc ggttcactat atgttaaacc aggtggaacc |
| 15481 | tcatcaggag atgccacaac tgcttatgct aatagtgttt ttaacatttg tcaagctgtc |
| 15541 | acggccaatg ttaatgcact tttatctact gatggtaaca aaattgccga taagtatgtc |
| 15601 | cgcaatttac aacacagact ttatgagtgt ctctatagaa atagagatgt tgacacagac |
| 15661 | tttgtgaatg agttttacgc atatttgcgt aaacatttct caatgatgat actctctgac |
| 15721 | gatgctgttg tgtgtttcaa tagcacttat gcatctcaag gtctagtggc tagcataaag |
| 15781 | aactttaagt cagttcttta ttatcaaaac aatgttttta tgtctgaagc aaaatgttgg |
| 15841 | actgagactg accttactaa aggacctcat gaattttgct ctcaacatac aatgctagtt |
| 15901 | aaacagggtg atgattatgt gtaccttcct tacccagatc catcaagaat cctaggggcc |
| 15961 | ggctgttttg tagatgatat cgtaaaaaca gatggtacac ttatgattga acggttcgtg |
| 16021 | tctttagcta tagatgctta cccacttact aaacatccta atcaggagta tgctgatgtc |
| 16081 | tttcatttgt acttacaata cataagaaag ctacatgatg agttaacagg acacatgtta |
| 16141 | gacatgtatt ctgttatgct tactaatgat aacacttcaa ggtattggga acctgagttt |
| 16201 | tatgaggcta tgtacacacc gcatacagtc ttacaggctg ttggggcttg tgttctttgc |
| 16261 | aattcacaga cttcattaag atgtggtgct tgcatacgta gaccattctt atgttgtaaa |
| 16321 | tgctgttacg accatgtcat atcaacatca cataaattag tcttgtctgt taatccgtat |
| 16381 | gtttgcaatg ctccaggttg tgatgtcaca gatgtgactc aactttactt aggaggtatg |
| 16441 | agctattatt gtaaatcaca taaaccaccc attagttttc cattgtgtgc taatggacaa |
| 16501 | gtttttggtt tatataaaaa tacatgtgtt ggtagcgata atgttactga ctttaatgca |
| 16561 | attgcaacat gtgactggac aaatgctggt gattacattt tagctaacac ctgtactgaa |
| 16621 | agactcaagc tttttgcagc agaaacgctc aaagctactg aggagacatt taaactgtct |
| 16681 | tatggtattg ctactgtacg tgaagtgctg tctgacagag aattacatct ttcatgggaa |
| 16741 | gttggtaaac ctagaccacc acttaaccga aattatgtct ttactggtta tcgtgtaact |
| 16801 | aaaaacagta aagtacaaat aggagagtac acctttgaaa aaggtgacta tggtgatgct |
| 16861 | gttgtttacc gaggtacaac aacttacaaa ttaaatgttg gtgattattt tgtgctgaca |
| 16921 | tcacatacag taatgccatt aagtgcacct acactagtgc cacaagagca ctatgttaga |
| 16981 | attactggct tatacccaac actcaatatc tcagatgagt tttctagcaa tgttgcaaat |
| 17041 | tatcaaaagg ttggtatgca aaagtattct acactccagg gaccacctgg tactggtaag |
| 17101 | agtcattttg ctattggcct agctctctac tacccttctg ctcgcatagt gtatacagct |
| 17161 | tgctctcatg ccgctgttga tgcactatgt gagaaggcat taaaatattt gcctatagat |
| 17221 | aaatgtagta gaattatacc tgcacgtgct cgtgtagagt gttttgataa attcaaagtg |
| 17281 | aattcaacat tagaacagta tgtcttttgt actgtaaatg cattgcctga gacgacagca |
| 17341 | gatatagttg tctttgatga aatttcaatg gccacaaatt atgatttgag tgttgtcaat |
| 17401 | gccagattac gtgctaagca ctatgtgtac attggcgacc ctgctcaatt acctgcacca |
| 17461 | cgcacattgc taactaaggg cacactagaa ccagaatatt tcaattcagt gtgtagactt |
| 17521 | atgaaaacta taggtccaga catgttcctc ggaacttgtc ggcgttgtcc tgctgaaatt |
| 17581 | gttgacactg tgagtgcttt ggtttatgat aataagctta aagcacataa agacaaatca |
| 17641 | gctcaatgct ttaaaatgtt ttataagggt gttatcacgc atgatgtttc atctgcaatt |
| 17701 | aacaggccac aaataggcgt ggtaagagaa ttccttacac gtaaccctgc ttggagaaaa |
| 17761 | gctgtcttta tttcacctta taattcacag aatgctgtag cctcaaagat tttgggacta |
| 17821 | ccaactcaaa ctgttgattc atcacagggc tcagaatatg actatgtcat attcactcaa |
| 17881 | accactgaaa cagctcactc ttgtaatgta aacagattta atgttgctat taccagagca |
| 17941 | aaagtaggca tactttgcat aatgtctgat agagaccttt atgacaagtt gcaatttaca |
| 18001 | agtcttgaaa ttccacgtag gaatgtggca actttacaag ctgaaaatgt aacaggactc |
| 18061 | tttaaagatt gtagtaaggt aatcactggg ttacatccta cacaggcacc tacacacctc |
| 18121 | agtgttgaca ctaaattcaa aactgaaggt ttatgtgttg acatacctgg catacctaag |
| 18181 | gacatgacct atagaagact catctctatg atgggtttta aaatgaatta tcaagttaat |
| 18241 | ggttacccta acatgtttat cacccgcgaa gaagctataa gacatgtacg tgcatggatt |
| 18301 | ggcttcgatg tcgaggggtg tcatgctact agagaagctg ttggtaccaa tttaccttta |
| 18361 | cagctaggtt tttctacagg tgttaaccta gttgctgtac ctacaggtta tgttgataca |
| 18421 | cctaataata cagatttttc cagagttagt gctaaaccac cgcctggaga tcaatttaaa |
| 18481 | cacctcatac cacttatgta caaaggactt ccttggaatg tagtgcgtat aaagattgta |
| 18541 | caaatgttaa gtgacacact taaaaatctc tctgacagag tcgtatttgt cttatgggca |
| 18601 | catggctttg agttgacatc tatgaagtat tttgtgaaaa taggacctga gcgcacctgt |
| 18661 | tgtctatgtg atagacgtgc cacatgcttt tccactgctt cagacactta tgcctgttgg |
| 18721 | catcattcta ttggatttga ttacgtctat aatccgttta tgattgatgt tcaacaatgg |
| 18781 | ggttttacag gtaacctaca aagcaaccat gatctgtatt gtcaagtcca tggtaatgca |
| 18841 | catgtagcta gttgtgatgc aatcatgact aggtgtctag ctgtccacga gtgctttgtt |
| 18901 | aagcgtgttg actggactat tgaatatcct ataattggtg atgaactgaa gattaatgcg |
| 18961 | gcttgtagaa aggttcaaca catggttgtt aaagctgcat tattagcaga caaattccca |
| 19021 | gttcttcacg acattggtaa ccctaaagct attaagtgtg tacctcaagc tgatgtagaa |
| 19081 | tggaagttct atgatgcaca gccttgtagt gacaaagctt ataaaataga agaattattc |
| 19141 | tattcttatg ccacacattc tgacaaattc acagatggtg tatgcctatt ttggaattgc |
| 19201 | aatgtcgata gatatcctgc taattccatt gtttgtagat ttgacactag agtgctatct |
| 19261 | aaccttaact tgcctggttg tgatggtggc agtttgtatg taaataaaca tgcattccac |
| 19321 | acaccagctt ttgataaaag tgcttttgtt aatttaaaac aattaccatt tttctattac |
| 19381 | tctgacagtc catgtgagtc tcatggaaaa caagtagtgt cagatataga ttatgtacca |
| 19441 | ctaaagtctg ctacgtgtat aacacgttgc aatttaggtg gtgctgtctg tagacatcat |
| 19501 | gctaatgagt acagattgta tctcgatgct tataacatga tgatctcagc tggctttagc |
| 19561 | ttgtgggttt acaaacaatt tgatacttat aacctctgga acacttttac aagacttcag |
| 19621 | agtttagaaa atgtggcttt taatgttgta aataagggac actttgatgg acaacagggt |
| 19681 | gaagtaccag tttctatcat taataacact gtttacacaa aagttgatgg tgttgatgta |
| 19741 | gaattgtttg aaaataaaac aacattacct gttaatgtag catttgagct ttgggctaag |
| 19801 | cgcaacatta aaccagtacc agaggtgaaa atactcaata atttgggtgt ggacattgct |
| 19861 | gctaatactg tgatctggga ctacaaaaga gatgctccag cacatatatc tactattggt |
| 19921 | gtttgttcta tgactgacat agccaagaaa ccaactgaaa cgatttgtgc accactcact |
| 19981 | gtcttttttg atggtagagt tgatggtcaa gtagacttat ttagaaatgc ccgtaatggt |
| 20041 | gttcttatta cagaaggtag tgttaaaggt ttacaaccat ctgtaggtcc caaacaagct |
| 20101 | agtcttaatg gagtcacatt aattggagaa gccgtaaaaa cacagttcaa ttattataag |
| 20161 | aaagttgatg gtgttgtcca acaattacct gaaacttact ttactcagag tagaaattta |
| 20221 | caagaattta aacccaggag tcaaatggaa attgatttct tagaattagc tatggatgaa |
| 20281 | ttcattgaac ggtataaatt agaaggctat gccttcgaac atatcgttta tggagatttt |
| 20341 | agtcatagtc agttaggtgg tttacatcta ctgattggac tagctaaacg ttttaaggaa |
| 20401 | tcaccttttg aattagaaga ttttattcct atggacagta cagttaaaaa ctatttcata |
| 20461 | acagatgcgc aaacaggttc atctaagtgt gtgtgttctg ttattgattt attacttgat |
| 20521 | gattttgttg aaataataaa atcccaagat ttatctgtag tttctaaggt tgtcaaagtg |
| 20581 | actattgact atacagaaat ttcatttatg ctttggtgta aagatggcca tgtagaaaca |
| 20641 | ttttacccaa aattacaatc tagtcaagcg tggcaaccgg gtgttgctat gcctaatctt |
| 20701 | tacaaaatgc aaagaatgct attagaaaag tgtgaccttc aaaattatgg tgatagtgca |
| 20761 | acattaccta aaggcataat gatgaatgtc gcaaaatata ctcaactgtg tcaatattta |
| 20821 | aacacattaa cattagctgt accctataat atgagagtta tacattttgg tgctggttct |
| 20881 | gataaaggag ttgcaccagg tacagctgtt ttaagacagt ggttgcctac gggtacgctg |
| 20941 | cttgtcgatt cagatcttaa tgactttgtc tctgatgcag attcaacttt gattggtgat |
| 21001 | tgtgcaactg tacatacagc taataaatgg gatctcatta ttagtgatat gtacgaccct |
| 21061 | aagactaaaa atgttacaaa agaaaatgac tctaaagagg gttttttcac ttacatttgt |
| 21121 | gggtttatac aacaaaagct agctcttgga ggttccgtgg ctataaagat aacagaacat |
| 21181 | tcttggaatg ctgatcttta taagctcatg ggacacttcg catggtggac agcctttgtt |
| 21241 | actaatgtga atgcgtcatc atctgaagca tttttaattg gatgtaatta tcttggcaaa |
| 21301 | ccacgcgaac aaatagatgg ttatgtcat catgcaaatt acatattttg gaggaataca |
| 21361 | aatccaattc agttgtcttc ctattcttta tttgacatga gtaaatttcc ccttaaatta |
| 21421 | aggggtactg ctgttatgtc tttaaaagaa ggtcaaatca atgatatgat tttatctctt |
| 21481 | cttagtaaag gtagacttat aattagagaa aacaacagag ttgttatttc tagtgatgtt |
| 21541 | cttgttaaca actaaacgaa caatgtttgt ttttcttgtt ttattgccac tagtctctag |
| 21601 | tcagtgtgtt aatcttacaa ccagaactca attaccccct gcatacacta attctttcac |
| 21661 | acgtggtgtt tattaccctg acaaagtttt cagatcctca gttttacatt caactcagga |
| 21721 | cttgttctta cctttctttt ccaatgttac ttggttccat gctatacatg tctctgggac |
| 21781 | caatggtact aagaggtttg ataaccctgt cctaccattt aatgatggtg tttattttgc |
| 21841 | ttccactgag aagtctaaca taataagagg ctggattttt ggtactactt tagattcgaa |
| 21901 | gacccagtcc ctacttattg ttaataacgc tactaatgtt gttattaaag tctgtgaatt |
| 21961 | tcaattttgt aatgatccat ttttgggtgt ttattaccac aaaaacaaca aaagttggat |
| 22021 | ggaaagtgag ttcagagttt attctagtgc gaataattgc acttttgaat atgtctctca |
| 22081 | gccttttctt atggaccttg aaggaaaaca gggtaatttc aaaaatctta gggaatttgt |
| 22141 | gtttaagaat attgatggtt attttaaaat atattctaag cacacgccta ttaatttagt |
| 22201 | gcgtgatctc cctcagggtt tttcggcttt agaaccattg gtagatttgc caataggtat |
| 22261 | taacatcact aggtttcaaa ctttacttgc tttacataga agttatttga ctcctggtga |
| 22321 | ttcttcttca ggttggacag ctggtgctgc agcttattat gtgggttatc ttcaacctag |
| 22381 | gacttttcta ttaaaatata atgaaaatgg aaccattaca gatgctgtag actgtgcact |
| 22441 | tgaccctctc tcagaaacaa agtgtacgtt gaaatccttc actgtagaaa aaggaatcta |
| 22501 | tcaaacttct aactttagag tccaaccaac agaatctatt gttagatttc ctaatattac |
| 22561 | aaacttgtgc ccttttggtg aagtttttaa cgccaccaga tttgcatctg tttatgcttg |
| 22621 | gaacaggaag agaatcagca actgtgttgc tgattattct gtcctatata attccgcatc |
| 22681 | attttccact tttaagtgtt atggagtgtc tcctactaaa ttaaatgatc tctgctttac |
| 22741 | taatgtctat gcagattcat ttgtaattag aggtgatgaa gtcagacaaa tcgctccagg |
| 22801 | gcaaactgga aagattgctg attataatta taaattacca gatgatttta caggctgcgt |
| 22861 | tatagcttgg aattctaaca atcttgattc taaggttggt ggtaattata attacctgta |
| 22921 | tagattgttt aggaagtcta atctcaaacc ttttgagaga gatatttcaa ctgaaatcta |
| 22981 | tcaggccggt agcacacctt gtaatggtgt tgaaggtttt aattgttact ttcctttaca |
| 23041 | atcatatggt ttccaaccca ctaatggtgt tggttaccaa ccatacagag tagtagtact |
| 23101 | ttcttttgaa cttctacatg caccagcaac tgtttgtgga cctaaaaagt ctactaattt |
| 23161 | ggttaaaaac aaatgtgtca atttcaactt caatggttta acaggcacag gtgttcttac |
| 23221 | tgagtctaac aaaaagtttc tgcctttcca acaatttggc agagacattg ctgacactac |
| 23281 | tgatgctgtc cgtgatccac agacacttga gattcttgac attacaccat gttcttttgg |
| 23341 | tggtgtcagt gttataacac caggaacaaa tacttctaac caggttgctg ttctttatca |
| 23401 | ggatgttaac tgcacagaag tccctgttgc tattcatgca gatcaactta ctcctacttg |
| 23461 | gcgtgtttat tctacaggtt ctaatgtttt tcaaacacgt gcaggctgtt taataggggc |
| 23521 | tgaacatgtc aacaactcat atgagtgtga catacccatt ggtgcaggta tatgcgctag |
| 23581 | ttatcagact cagactaatt ctcctcggcg ggcacgtagt gtagctagtc aatccatcat |
| 23641 | tgcctacact atgtcacttg gtgcagaaaa ttcagttgct tactctaata actctattgc |
| 23701 | catacccaca aattttacta ttagtgttac cacagaaatt ctaccagtgt ctatgaccaa |
| 23761 | gacatcagta gattgtacaa tgtacatttg tggtgattca actgaatgca gcaatctttt |
| 23821 | gttgcaatat ggcagttttt gtacacaatt aaaccgtgct ttaactggaa tagctgttga |
| 23881 | acaagacaaa aacacccaag aagtttttgc acaagtcaaa caaatttaca aaacaccacc |
| 23941 | aattaaagat tttggtggtt ttaatttttc acaaatatta ccagatccat caaaaccaag |
| 24001 | caagaggtca tttattgaag atctactttt caacaaagtg acacttgcag atgctggctt |
| 24061 | catcaaacaa tatggtgatt gccttggtga tattgctgct agagacctca tttgtgcaca |
| 24121 | aaagtttaac ggccttactg ttttgccacc tttgctcaca gatgaaatga ttgctcaata |
| 24181 | cacttctgca ctgttagcgg gtacaatcac ttctggttgg acctttggtg caggtgctgc |
| 24241 | attacaaata ccatttgcta tgcaaatggc ttataggttt aatggtattg gagttacaca |
| 24301 | gaatgttctc tatgagaacc aaaaattgat tgccaaccaa tttaatagtg ctattggcaa |
| 24361 | aattcaagac tcactttctt ccacagcaag tgcacttgga aaacttcaag atgtggtcaa |
| 24421 | ccaaaatgca caagctttaa acacgcttgt taaacaactt agctccaatt ttggtgcaat |
| 24481 | ttcaagtgtt ttaaatgata tcctttcacg tcttgacaaa gttgaggctg aagtgcaaat |
| 24541 | tgataggttg atcacaggca gacttcaaag tttgcagaca tatgtgactc aacaattaat |
| 24601 | tagagctgca gaaatcagag cttctgctaa tcttgctgct actaaaatgt cagagtgtgt |
| 24661 | acttggacaa tcaaaaagag ttgatttttg tggaaagggc tatcatctta tgtccttccc |
| 24721 | tcagtcagca cctcatggtg tagtcttctt gcatgtgact tatgtccctg cacaagaaaa |
| 24781 | gaacttcaca actgctcctg ccatttgtca tgatggaaaa gcacactttc ctcgtgaagg |
| 24841 | tgtctttgtt tcaaatggca cacactggtt tgtaacacaa aggaattttt atgaaccaca |
| 24901 | aatcattact acagacaaca catttgtgtc tggtaactgt gatgttgtaa taggaattgt |
| 24961 | caacaacaca gtttatgatc ctttgcaacc tgaattagac tcattcaagg aggagttaga |
| 25021 | taaatatttt aagaatcata catcaccaga tgttgattta ggtgacatct ctggcattaa |
| 25081 | tgcttcagtt gtaaacattc aaaaagaaat tgaccgcctc aatgaggttg ccaagaattt |
| 25141 | aaatgaatct ctcatcgatc tccaagaact tggaaagtat gagcagtata taaaatggcc |
| 25201 | atggtacatt tggctaggtt ttatagctgg cttgattgcc atagtaatgg tgacaattat |
| 25261 | gctttgctgt atgaccagtt gctgtagttg tctcaagggc tgttgttctt gtggatcctg |
| 25321 | ctgcaaattt gatgaagacg actctgagcc agtgctcaaa ggagtcaaat tacattacac |
| 25381 | ataaacgaac ttatggattt gtttatgaga atcttcacaa ttggaactgt aactttgaag |
| 25441 | caaggtgaaa tcaaggatgc tactccttca gattttgttc gcgctactgc aacgataccg |
| 25501 | atacaagcct cactcccttt cggatggctt attgttggcg ttgcacttct tgctgttttt |
| 25561 | cagagcgctt ccaaaatcat aaccctcaaa aagagatggc aactagcact ctccaagggt |
| 25621 | gttcactttg tttgcaactt gctgttgttg tttgtaacag tttactcaca ccttttgctc |
| 25681 | gttgctgctg gccttgaagc cccttttctc tatctttatg ctttagtcta cttcttgcag |
| 25741 | agtataaact ttgtaagaat aataatgagg ctttggcttt gctggaaatg ccgttccaaa |
| 25801 | aacccattac tttatgatgc caactatttt ctttgctggc atactaattg ttacgactat |
| 25861 | tgtatacctt acaatagtgt aacttcttca attgtcatta cttcaggtga tggcacaaca |
| 25921 | agtcctattt ctgaacatga ctaccagatt ggtggttata ctgaaaaatg ggaatctgga |
| 25981 | gtaaaagact gtgttgtatt acacagttac ttcacttcag actattacca gctgtactca |
| 26041 | actcaattga gtacagacac tggtgttgaa catgttacct tcttcatcta caataaaatt |
| 26101 | gttgatgagc ctgaagaaca tgtccaaatt cacacaatcg acggttcatc cggagttgtt |
| 26161 | aatccagtaa tggaaccaat ttatgatgaa ccgacgacga ctactagcgt gcctttgtaa |
| 26221 | gcacaagctg atgagtacga acttatgtac tcattcgttt cggaagagac aggtacgtta |
| 26281 | atagttaata gcgtacttct ttttcttgct ttcgtggtat tcttgctagt tacactagcc |
| 26341 | atccttactg cgcttcgatt gtgtgcgtac tgctgcaata ttgttaacgt gagtcttgta |
| 26401 | aaaccttctt tttacgttta ctctcgtgtt aaaaatctga attcttctag agttcctgat |
| 26461 | cttctggtct aaacgaacta aatattatat tagtttttct gtttggaact ttaattttag |
| 26521 | ccatggcaga ttccaacggt actattaccg ttgaagagct taaaaagctc cttgaacaat |
| 26581 | ggaacctagt aataggtttc ctattcctta catggatttg tcttctacaa tttgcctatg |
| 26641 | ccaacaggaa taggtttttg tatataatta agttaatttt cctctggctg ttatggccag |
| 26701 | taactttagc ttgttttgtg cttgctgctg tttacagaat aaattggatc accggtggaa |
| 26761 | ttgctatcgc aatggcttgt cttgtaggct tgatgtggct cagctacttc attgcttctt |
| 26821 | tcagactgtt tgcgcgtacg cgttccatgt ggtcattcaa tccagaaact aacattcttc |
| 26881 | tcaacgtgcc actccatggc actattctga ccagaccgct tctagaaagt gaactcgtaa |
| 26941 | tcggagctgt gatccttcgt ggacatcttc gtattgctgg acaccatcta ggacgctgtg |
| 27001 | acatcaagga cctgcctaaa gaaatcactg ttgctacatc acgaacgctt tcttattaca |
| 27061 | aattgggagc ttcgcagcgt gtagcaggtg actcaggttt tgctgcatac agtcgctaca |
| 27121 | ggattggcaa ctataaatta aacacagacc attccagtag cagtgacaat attgctttgc |
| 27181 | ttgtacagta agtgacaaca gatgtttcat ctcgttgact ttcaggttac tatagcagag |
| 27241 | atattactaa ttattatgag gacttttaaa gtttccattt ggaatcttga ttacatcata |
| 27301 | aacctcataa ttaaaaattt atctaagtca ctaactgaga ataaatattc tcaattagat |
| 27361 | gaagagcaac caatggagat tgattaaacg aacatgaaaa ttattctttt cttggcactg |
| 27421 | ataacactcg ctacttgtga gctttatcac taccaagagt gtgttagagg tacaacagta |
| 27481 | cttttaaaag aaccttgctc ttctggaaca tacgagggca attcaccatt tcatcctcta |
| 27541 | gctgataaca aatttgcact gacttgcttt agcactcaat ttgcttttgc ttgtcctgac |
| 27601 | ggcgtaaaac acgtctatca gttacgtgcc agatcagttt cacctaaact gttcatcaga |
| 27661 | caagaggaag ttcaagaact ttactctcca atttttctta ttgttgcggc aatagtgttt |
| 27721 | ataacacttt gcttcacact caaaagaaag acagaatgat tgaactttca ttaattgact |
| 27781 | tctatttgtg ctttttagcc tttctgctat tccttgtttt aattatgctt attatctttt |
| 27841 | ggttctcact tgaactgcaa gatcataatg aaacttgtca cgcctaaacg aacatgaaat |
| 27901 | ttcttgtttt cttaggaatc atcacaactg tagctgcatt tcaccaagaa tgtagtttac |
| 27961 | agtcatgtac tcaacatcaa ccatatgtag ttgatgaccc gtgtcctatt cacttctatt |
| 28021 | ctaaatggta tattagagta ggagctagaa aatcagcacc tttaattgaa ttgtgcgtgg |
| 28081 | atgaggctgg ttctaaatca cccattcagt acatcgatat cggtaattat acagtttcct |
| 28141 | gtttaccttt tacaattaat tgccaggaac ctaaattggg tagtcttgta gtgcgttgtt |
| 28201 | cgttctatga agacttttta gagtatcatg acgttcgtgt tgttttagat ttcatctaaa |
| 28261 | cgaacaaact aaaatgtctg ataatggacc ccaaaatcag cgaaatgcac cccgcattac |
| 28321 | gtttggtgga ccctcagatt caactggcag taaccagaat ggagaacgca gtggggcgcg |
| 28381 | atcaaaacaa cgtcggcccc aaggtttacc caataatact gcgtcttggt tcaccgctct |
| 28441 | cactcaacat ggcaaggaag accttaaatt ccctcgagga caaggcgttc caattaacac |
| 28501 | caatagcagt ccagatgacc aaattggcta ctaccgaaga gctaccagac gaattcgtgg |
| 28561 | tggtgacggt aaaatgaaag atctcagtcc aagatggtat ttctactacc taggaactgg |
| 28621 | gccagaagct ggacttccct atggtgctaa caaagacggc atcatatggg ttgcaactga |
| 28681 | gggagccttg aatacaccaa aagatcacat tggcacccgc aatcctgcta acaatgctgc |
| 28741 | aatcgtgcta caacttcctc aaggaacaac attgccaaaa ggcttctacg cagaagggag |
| 28801 | cagaggcggc agtcaagcct cttctcgttc ctcatcacgt agtcgcaaca gttcaagaaa |
| 28861 | ttcaactcca ggcagcagta ggggaacttc tcctgctaga atggctggca atggcggtga |
| 28921 | tgctgctctt gctttgctgc tgcttgacag attgaaccag cttgagagca aaatgtctgg |
| 28981 | taaaggccaa caacaacaag gccaaactgt cactaagaaa tctgctgctg aggcttctaa |
| 29041 | gaagcctcgg caaaaacgta ctgccactaa agcatacaat gtaacacaag ctttcggcag |
| 29101 | acgtggtcca gaacaaaccc aaggaaattt tggggaccag gaactaatca gacaaggaac |
| 29161 | tgattacaaa cattggccgc aaattgcaca atttgccccc agcgcttcag cgttcttcgg |
| 29221 | aatgtcgcgc attggcatgg aagtcacacc ttcgggaacg tggttgacct acacaggtgc |
| 29281 | catcaaattg gatgacaaag atccaaattt caaagatcaa gtcattttgc tgaataagca |
| 29341 | tattgacgca tacaaaacat tcccaccaac agagcctaaa aaggacaaaa agaagaaggc |
| 29401 | tgatgaaact caagccttac cgcagagaca gaagaaacag caaactgtga ctcttcttcc |
| 29461 | tgctgcagat ttggatgatt tctccaaaca attgcaacaa tccatgagca gtgctgactc |
| 29521 | aactcaggcc taaactcatg cagaccacac aaggcagatg ggctatataa acgttttcgc |
| 29581 | ttttccgttt acgatatata gtctactctt gtgcagaatg aattctcgta actacatagc |
| 29641 | acaagtagat gtagttaact ttaatctcac atagcaatct ttaatcagtg tgtaacatta |
| 29701 | gggaggactt gaaagagcca ccacattttc accgaggcca cgcggagtac gatcgagtgt |
| 29761 | acagtgaaca atgctaggga gagctgccta tatggaagag ccctaatgtg taaaattaat |
| 29821 | tttagtagtg ctatccccat gtgattttaa tagcttctta ggagaatgac aaaaaaaaaa |
| 29881 | aaaaaaaaaa aaaaaaaaaa aaa |

### General Definitions

The following definitions are included for the purpose of understanding the present subject matter and for constructing the appended patent claims. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994): Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

The term “disease” refers to any deviation from the normal health of a mammal and includes a state when disease symptoms are present, as well as conditions in which a deviation (e.g., SARS-COV2) has occurred, but symptoms are not yet manifested.

A viral infection, as used herein, may refer to a coronavirus (for example, SARS-COV2), hepatitis virus infection, an influenza virus infection, a herpes simplex virus infection, an enterovirus infection, a rotavirus infection, a dengue virus infection, a poxvirus infection, a human immunodeficiency virus infection, an adenovirus infection, a coronavirus infection, an arenavirus infection, a measles virus infection, a retrovirus infection or a Norovirus infection.

As used herein, the term “engineered immune cell” refers to an immune cell (e.g., T cell, NK cell, NKT cell, B cell, dendritic cell, myeloid cell, etc.) that is genetically modified. The term “chimeric antigen receptor” (“CAR”) refers to a recombinant polypeptide construct comprising at least an extracellular antigen binding domain and a transmembrane domain. Upon binding to their target (e.g., virus-infected cells), CARs typically modify the immune response of the immune cells on which they are displayed.

As used herein, an “immune response” refers to the action of a cell of the immune system (e.g., T lymphocytes, B lymphocytes, natural killer (NK) cells, macrophages, cosinophils, mast cells, dendritic cells, neutrophils, etc.) and soluble macromolecules produced by any of these cells or the liver (e.g., antibodies, cytokines, and complement) that results in selective targeting, binding to, damage to, destruction of, and/or elimination from a subject of invading pathogens (e.g., viruses), cells or tissues infected with pathogens, or cancerous cells or other abnormal/diseased-associated cells.

A “lectin” is a carbohydrate-binding protein which is widely found in nature, particularly plants. Lectins are highly specific for sugar moieties of other molecules and mediate attachment and binding of bacteria and viruses to target molecules in cells. Lectins have been used in many aspects of glycobiology (Andre' et al., Molecules, 20:1788-1823 (2015): Gabius et al., Trends Biochem. Sci., 36 (6): 298-313 (2011), and Gabius et al., Trends Biochem. Sci., 40:360-376 (2015)), including as potential antiviral agents.

“Patient” or “subject in need thereof” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

As used herein, the engineered immune cell is a natural killer (NK) cell. NK cells are a type of cytotoxic lymphocyte that plays a role in the innate immune system. NK cells are defined as large granular lymphocytes and constitute the third kind of cells differentiated from the common lymphoid progenitor which also gives rise to B and T lymphocytes (see, e.g., Immunobiology, 5th ed., Janeway et al., eds., Garland Publishing, New York, N.Y. (2001)). NK cells differentiate and mature in the bone marrow, lymph node, spleen, tonsils, and thymus. Following maturation, NK cells enter into the circulation as large lymphocytes with distinctive cytotoxic granules. NK cells are able to recognize and kill some abnormal cells, such as, for example, some tumor cells and virus-infected cells, and are thought to be important in the innate immune defense against intracellular pathogens. With respect to T cells, the NK cell can be a cultured NK cell or an NK cell obtained directly from a mammal. If obtained from a mammal, the NK cell can be obtained from numerous sources, including but not limited to blood, bone marrow, lymph node, the thymus, or other tissues or fluids. NK cells can also be enriched for or purified. The NK cell desirably is a human NK cell. NK cell lines are available from, e.g., the American Type Culture Collection (ATCC, Manassas, Va.) and include, for example, NK-92 cells (ATCC CRL-2407), NK92MI cells (ATCC CRL-2408), and derivatives thereof.

When T cells or NK cells are administered to a mammal, the cells can be allogeneic or autologous to the mammal. In “autologous” administration methods, cells (e.g., immune cells (lymphocytes))) are removed from a mammal, stored, engineered or modified (as described herein) and returned back to the same mammal. In “allogeneic” administration methods, a mammal receives cells (e.g., blood-forming stem cells or lymphocytes) from a genetically similar, but not identical, donor.

The terms “subject,” “patient,” “individual,” etc. are not intended to be limiting and can be generally interchanged. That is, an individual described as a “patient” does not necessarily have a given disease but may be merely seeking medical advice.

The transitional term “comprising.” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B:” “one or more of A and B:” and “A and/or B” are each intended to mean “A alone, B alone, or A and B together.” A similar interpretation is also intended for lists including three or more items. For example, the phrases “at least one of A, B, and C:” “one or more of A, B, and C:” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg. 0.5 mg. 0.6 mg etc. up to and including 5.0 mg.

As used in the description herein and throughout the claims that follow, the meaning of “a.” “an,” and “the” includes plural reference unless the context clearly dictates otherwise.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

As used herein, the terms “treat” and “prevent” are not intended to be absolute terms. In various embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” etc. refer to the amount of an agent that is sufficient to achieve a desired effect, as described herein. In embodiments, the term “effective” when referring to an amount of cells or a therapeutic compound may refer to a quantity of the cells or the compound that is sufficient to yield an improvement or a desired therapeutic response without undue adverse side effects (such as toxicity, irritation, or allergic response) commensurate with a reasonable benefit/risk ratio when used in the manner of this disclosure. In embodiments, the term “effective” when referring to the generation of a desired cell population may refer to an amount of one or more compounds that is sufficient to result in or promote the production of members of the desired cell population, especially compared to culture conditions that lack the one or more compounds.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (RNA or DNA) is free of the genes or sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated. A purified or isolated polynucleotide (ribonucleic acid (RNA) or deoxyribonucleic acid (DNA)) is free of the genes or sequences that flank it in its natural-occurring state. A purified or isolated polypeptide is free of the amino acids or sequences that flank it in its naturally-occurring state. Purified also defines a degree of sterility that is safe for administration to a human subject, e.g., lacking infectious or toxic agents. In the case of tumor antigens, the antigen may be purified or a processed preparation such as a tumor cell lysate.

A “control” sample or value refers to a sample that serves as a reference, usually a known reference, for comparison to a test sample. For example, a test sample can be taken from a test subject, e.g., a subject with a coronavirus (e.g., SARS-COV2), and compared to samples from known conditions, e.g., a subject (or subjects) that does not have a coronavirus (e.g., SARS-COV2) (a negative or normal control), or a subject (or subjects) who does have a coronavirus (e.g., SARS-COV2) (positive control). A control can also represent an average value gathered from a number of tests or results. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are variable in controls, variation in test samples will not be considered as significant.

The term, “normal amount” with respect to a compound (e.g., a protein or mRNA) refers to a normal amount of the compound in an individual who does not have a coronavirus (e.g., SARS-COV2) in a healthy or general population. The amount of a compound can be measured in a test sample and compared to the “normal control” level, utilizing techniques such as reference limits, discrimination limits, or risk defining thresholds to define cutoff points and abnormal values (e.g., for a coronavirus (e.g., SARS-COV2) or a symptom thereof). The normal control level means the level of one or more compounds or combined compounds typically found in a subject known not suffering from a coronavirus (e.g., SARS-COV2). Such normal control levels and cutoff points may vary based on whether a compound is used alone or in a formula combining with other compounds into an index. Alternatively, the normal control level can be a database of compounds patterns from previously tested subjects who did not develop a coronavirus (e.g., SARS-COV2) or a particular symptom thereof (e.g., in the event the coronavirus (e.g., SARS-COV2) develops or a subject already having a coronavirus (e.g., SARS-COV2) is tested) over a clinically relevant time horizon.

The level that is determined may be the same as a control level or a cut off level or a threshold level or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease (or a symptom thereof) in question or is not at risk for the disease.

Relative to a control level, the level that is determined may an increased level. As used herein, the term “increased” with respect to level (e.g., protein or mRNA level) refers to any % increase above a control level. In various embodiments, the increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level. As used herein, the term “decreased” with respect to level (e.g., protein or mRNA level) refers to any % decrease below a control level. In various embodiments, the decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

The terms “polypeptide,” “peptide” and “protein” are used interchangeably herein to refer to a polymer of amino acid residues, wherein the polymer may in embodiments be conjugated to a moiety that does not consist of amino acids. The terms also apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed or chemically synthesized as a single moiety.

“Polypeptide fragment” refers to a polypeptide that has an amino-terminal and/or carboxy-terminal deletion, in which the remaining amino acid sequence is usually identical to the corresponding positions in the naturally-occurring sequence. Fragments typically are at least 5, 6, 8 or 10 amino acids long, at least 14 amino acids long, at least 20 amino acids long, at least 50 amino acids long, or at least 70 amino acids long.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. In embodiments, two sequences are 100% identical. In embodiments, two sequences are 100% identical over the entire length of one of the sequences (e.g., the shorter of the two sequences where the sequences have different lengths). In embodiments, identity may refer to the complement of a test sequence. In embodiments, the identity exists over a region that is at least about 10 to about 100, about 20 to about 75, about 30 to about 50 amino acids or nucleotides in length. In embodiments, the identity exists over a region that is at least about 50 amino acids or nucleotides in length, or more preferably over a region that is 100 to 500, 100 to 200, 150 to 200, 175 to 200, 175 to 225, 175 to 250, 200 to 225, 200 to 250 or more amino acids or nucleotides in length.

For sequence comparison, typically one sequence acts as a reference sequence, to which test sequences are compared. In embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In embodiments, a comparison window is the entire length of one or both of two aligned sequences. In embodiments, two sequences being compared comprise different lengths, and the comparison window is the entire length of the longer or the shorter of the two sequences. In embodiments relating to two sequences of different lengths, the comparison window includes the entire length of the shorter of the two sequences. In embodiments relating to two sequences of different lengths, the comparison window includes the entire length of the longer of the two sequences.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see. e.g., Current Protocols in Molecular Biology (Ausubel et al., eds. 1995 supplement)).

Non-limiting examples of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information (NCBI), as is known in the art. An exemplary BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues: always >0) and N (penalty score for mismatching residues: always <0)). For amino acid sequences, a scoring matrix is used to calculate the cumulative score. Extension of the word hits in each direction are halted when: the cumulative alignment score falls off by the quantity X from its maximum achieved value: the cumulative score goes to zero or below; due to the accumulation of one or more negative-scoring residue alignments; or the end of either sequence is reached. The BLAST algorithm parameters W. T, and X determine the sensitivity and speed of the alignment. In embodiments, the NCBI BLASTN or BLASTP program is used to align sequences. In embodiments, the BLASTN or BLASTP program uses the defaults used by the NCBI. In embodiments, the BLASTN program (for nucleotide sequences) uses as defaults: a word size (W) of 28: an expectation threshold (E) of 10; max matches in a query range set to 0; match/mismatch scores of 1, −2: linear gap costs; the filter for low complexity regions used; and mask for lookup table only used. In embodiments, the BLASTP program (for amino acid sequences) uses as defaults: a word size (W) of 3: an expectation threshold (E) of 10: max matches in a query range set to 0); the BLOSUM62 matrix (see Henikoff & Henikoff, Proc. Natl. Acad. Sci. USA 89:10915 (1992)): gap costs of existence: 11 and extension: 1; and conditional compositional score matrix adjustment.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

“Nucleic acid” refers to nucleotides (e.g., deoxyribonucleotides, ribonucleotides, and 2′-modified nucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent, or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones: non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences

As may be used herein, the terms “nucleic acid,” “nucleic acid molecule.” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides and/or ribonucleotides, and/or analogs, derivatives or modifications thereof. Different polynucleotides may have different three-dimensional structures, and may perform various functions, known or unknown. Non-limiting examples of polynucleotides include genomic DNA, a genome, mitochondrial DNA, a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

The term “amino acid residue,” as used herein, encompasses both naturally-occurring amino acids and non-naturally-occurring amino acids. Examples of non-naturally occurring amino acids include, but are not limited to, D-amino acids (i.e. an amino acid of an opposite chirality to the naturally-occurring form), N-α-methyl amino acids, C-α-methyl amino acids, β-methyl amino acids and D- or L-β-amino acids. Other non-naturally occurring amino acids include, for example, β-alanine (β-Ala), norleucine (Nle), norvaline (Nva), homoarginine (Har), 4-aminobutyric acid (γ-Abu), 2-aminoisobutyric acid (Aib), 6-aminohexanoic acid (8-Ahx), ornithine (orn), sarcosine, α-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine.

As used herein, “peptide bond” can be a naturally-occurring peptide bond or a non-naturally occurring (i.e. modified) peptide bond. Examples of suitable modified peptide bonds are well known in the art and include, but are not limited to, —CH₂NH—, —CH₂S—, —CH₂CH₂—, —CH═CH-(cis or trans), —COCH₂—, —CH(OH) CH₂—, —CH₂SO—, —CS—NH— and —NH—CO— (i.e. a reversed peptide bond) (see, for example, Spatola, Vega Data Vol. 1, Issue 3, (1983): Spatola, in Chemistry and Biochemistry of Amino Acids Peptides and Proteins, Weinstein, ed., Marcel Dekker, New York, p. 267 (1983); Morley, J. S., Trends Pharm. Sci. pp. 463-468 (1980); Hudson et al., Int. J. Pept. Prot. Res. 14:177-185 (1979); Spatola et al., Life Sci. 38:1243-1249 (1986): Hann, J. Chem. Soc. Perkin Trans. 1 307-314 (1982); Almquist et al., J. Med. Chem. 23:1392-1398 (1980): Jennings-White et al., Tetrahedron Lett. 23:2533 (1982); Szelke et al., EP 45665 (1982); Holladay et al., Tetrahedron Lett. 24:4401-4404 (1983); and Hruby, Life Sci. 31:189-199 (1982)).

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule: alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

### Methods for Treating a Coronavirus, e.g., SARS-CoV-2 and Other Viral Infections

Included herein is a method of preventing or treating a coronavirus, e.g., SARS-CoV-2 in a subject in need thereof. In further embodiments, the method comprises administering to the subject an effective amount of the composition comprising engineered NK cells expressing a CAR comprising an extracellular banana lectin domain, e.g., an H84T-BanLec domain. For example, methods for preventing or treating coronavirus, e.g., SARS-CoV-2, include administering a composition comprising engineered NK cells expressing a CAR comprising an extracellular banana lectin domain, e.g., an H84T-BanLec domain.

In other embodiments, the methods for treating coronavirus, e.g., SARS-CoV-2 comprise administering to a subject a composition comprising engineered NK cells expressing a CAR comprising an extracellular banana lectin domain, e.g., an H84T-BanLec domain produced according to the methods described herein, in combination with methods for controlling the outset of symptoms. In particular, the combination treatment can include administering readily known treatments. Additionally, combination therapy may include antivirals.

The present methods and compositions also may be utilized in the same manner disclosed herein to treat subjects and cells infected with or susceptible to infection with other coronaviruses such as Middle East Respiratory Syndrome coronavirus (MERS-COV) and/or,

SARS-coronavirus (SARS-CoV) (severe acute respiratory syndrome). Thus for example, methods are provided for treating a infection of Middle East Respiratory Syndrome coronavirus (MERS-COV) and/or SARS-coronavirus (SARS-CoV) that comprise administering to a subject or cells in need thereof (e.g. a subject or cells infected with Middle East Respiratory Syndrome coronavirus (MERS-COV) and/or SARS-coronavirus (SARS-CoV)) a composition comprising engineered NK cells expressing a CAR comprising an extracellular banana lectin domain, e.g., an H84T-BanLec domain produced according to the methods described herein, in combination with methods for controlling the outset of symptoms. In particular, the combination treatment can include administering readily known treatments. Additionally, combination therapy may include antivirals.

The present methods and compositions also may be utilized in the same manner disclosed herein to treat subjects and cells infected with or susceptible to infection with retroviruses including HIV, SIV. SHIV and/or HTLV and/or lentiviruses. Thus for example, methods are provided for treating a retrovirus infection such as HIV, SIV, SHIV and/or HTLV and/or a lentivirus that comprise administering to a subject or cells in need thereof (e.g. a subject or cells infected with a retrovirus such as HIV, SIV, SHIV and/or HTLV and/or a lentivirus) a composition comprising engineered NK cells expressing a CAR comprising an extracellular banana lectin domain, e.g., an H84T-BanLec domain produced according to the methods described herein, in combination with methods for controlling the outset of symptoms. In particular, the combination treatment can include administering readily known treatments. Additionally, combination therapy may include antivirals.

The described composition can be administered as a pharmaceutically or physiologically acceptable preparation or composition containing a physiologically acceptable carrier, excipient, or diluent, and administered to the tissues of the recipient organism of interest, including humans and non-human animals.

The composition comprising engineered NK cells expressing a CAR comprising an extracellular banana lectin domain, e.g., an H84T-BanLec domain can be prepared by re-suspending in a suitable liquid or solution such as sterile physiological saline or other physiologically acceptable injectable aqueous liquids. The amounts of the components to be used in such compositions can be routinely determined by those having skill in the art.

In examples, for injectable administration, the composition (e.g., a composition comprising engineered NK cells expressing a CAR comprising an extracellular banana lectin domain, e.g., an H84T-BanLec domain) is in sterile solution or suspension or can be resuspended in pharmaceutically- and physiologically-acceptable aqueous or oleaginous vehicles, which may contain preservatives, stabilizers, and material for rendering the solution or suspension isotonic with body fluids (i.e. blood) of the recipient. Non-limiting examples of excipients suitable for use include water, phosphate buffered saline, pH 7.4, 0.15 M aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures. The amounts or quantities, as well as the routes of administration used, are determined on an individual basis, and correspond to the amounts used in similar types of applications or indications known to those of skill in the art.

In embodiments, a therapeutically effective amount of the composition (e.g., a composition comprising engineered NK cells expressing a CAR comprising an extracellular banana lectin domain, e.g., an H84T-BanLec domain) in humans can be any therapeutically effective amount. In one embodiment, the composition (e.g., a composition comprising engineered NK cells expressing a CAR comprising an extracellular banana lectin domain, e.g., an H84T-BanLec domain) is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

In an embodiment, the composition (e.g., a composition comprising engineered NK cells expressing a CAR comprising an extracellular banana lectin domain, e.g., an H84T-BanLec domain) is administered once a week, or once every two weeks, or once every 3 weeks or once every 4 weeks for at least 1 week, in some embodiments for 1 to 4 weeks, from 2 to 6 weeks, from 2 to 8 weeks, from 2 to 10 weeks, or from 2 to 12 weeks, 2 to 16 weeks, or longer (e.g., 1, 2, 3, 4, 5, 6, 7, 8, 9, 10, 11, 12, 36, 48, or more weeks).

Additional advantages of the methods described herein include that the composition comprising the engineered NK cells expressing a CAR comprising an extracellular banana lectin domain, e.g., an H84T-BanLec domain can be injected systemically, as opposed to local delivery. Additional advantages include that patients requiring treatment typically require at least 1 local injections, and the injections are about 7 days apart. The compositions and methods described herein provide that patients require about 1 injection(s), systemically. In some examples, the injections can be every week.

### Pharmaceutical Compositions and Formulations

The present invention provides pharmaceutical compositions comprising an effective amount of a composition (e.g., a composition comprising engineered NK cells expressing a chimeric antigen receptor (CAR) which comprises an extracellular banana lectin domain, e.g., an H84T-BanLec domain) and at least one pharmaceutically acceptable excipient or carrier, wherein the effective amount is as described above in connection with the methods of the invention.

In one embodiment, the composition (e.g., a composition comprising engineered NK cells expressing a CAR comprising an extracellular banana lectin domain, e.g., an H84T-BanLec domain) is further combined with at least one additional therapeutic agent in a single dosage form. In one embodiment, the at least one additional therapeutic agent comprises an antiviral agent.

Non-limiting examples of anti-viral agents that may be used in combination as described herein include Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

Additional examples of one or more therapeutic agents that may be used in combination as described herein include include r one or more of hyperimmune globulins, remdesivir, oseltamivir. Galidesivir (BCX4430. Immucillin-A). 3-Deazaneplanocin A (DZNep, C-c3 Ado). Favipiravir (T-705. Avigan), lopinavir: ritonavir, lopinavir/ritonavir (e.g. KALETRA), ribavirin, lopinavir/ritonavir/ribavirin, Recombinant human interferon α1β, Huaier (including Huaier Granule), Eculizumab (Soliris), Recombinant human angiotensin-converting enzyme 2 (rhACE2), Carrimycin, Umifenivir (Arbidol), chloroquine phosphate, T89 (Dantonic), Fingolimod (including Fingolimod 0.5 mg), N-acetylcysteine, N-acetylcysteine+Fuzheng Huayu Tablet, YinHu QingWen Decoction, LV-SMENP-DC vaccine and/or antigen-specific CTLs.

The term “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). Exemplary doses and dosages regimens for the compositions in methods of treating muscle diseases or disorders are described herein.

The pharmaceutical compositions can take any suitable form (e.g. liquids, aerosols, solutions, inhalants, mists, sprays; or solids, powders, ointments, pastes, creams, lotions, gels, patches and the like) for administration by any desired route (e.g. pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

In embodiments, the pharmaceutical composition comprises an injectable form.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present invention as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present invention can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value. All percentages and ratios used herein, unless otherwise indicated, are by weight.

A typical amount of cells (e.g., engineered immune cells) administered to a mammal (e.g., a human) can be, for example, in the range of one million to 100 billion cells; however, amounts below or above this exemplary range are within the scope of the disclosure. For example, the daily dose of cells can be about 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), preferably about 10 million to about 100 billion cells (e.g., about 20 million cells, about 30 million cells, about 40 million cells, about 60) million cells, about 70 million cells, about 80 million cells, about 90 million cells, about 10 billion cells, about 25 billion cells, about 50 billion cells, about 75 billion cells, about 90) billion cells, or a range defined by any two of the foregoing values), more preferably about 100 million cells to about 50 billion cells (e.g., about 120 million cells, about 250 million cells, about 350) million cells, about 450 million cells, about 650 million cells, about 800 million cells, about 900 million cells, about 3 billion cells, about 30 billion cells, about 45 billion cells, or a range defined by any two of the foregoing values).

Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### Kits Comprising the Composition Comprising Engineered NK Cells Expressing a CAR Comprising an Extracellular Banana Lectin Domain, e.g., an H84T-BanLec Domain

In aspects, a kit for producing an engineered NK cell expressing a CAR comprising an extracellular banana lectin domain (e.g., an H84T-BanLec domain) is provided. In embodiments, the kit comprises the engineered NK cell and reagents.

In embodiments, the composition in the kit is suitable for delivery (e.g., local injection) to a subject.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention (e.g., a coronavirus, e.g., SARS-COV2), one or more syringes, one or more applicators, or a sterile solution suitable for reconstituting a pharmaceutical composition of the present invention.

### EXAMPLES

The following examples illustrate certain specific embodiments of the invention and are not meant to limit the scope of the invention. Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1: H84T BanLec CAR-T Cells

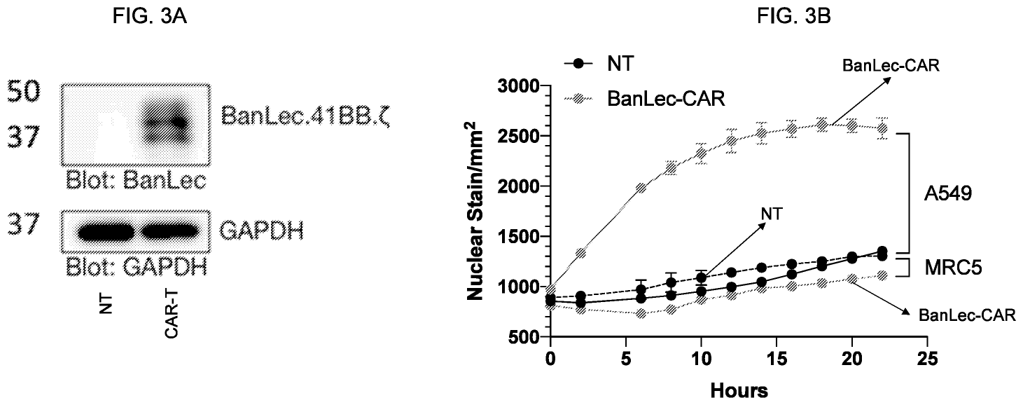

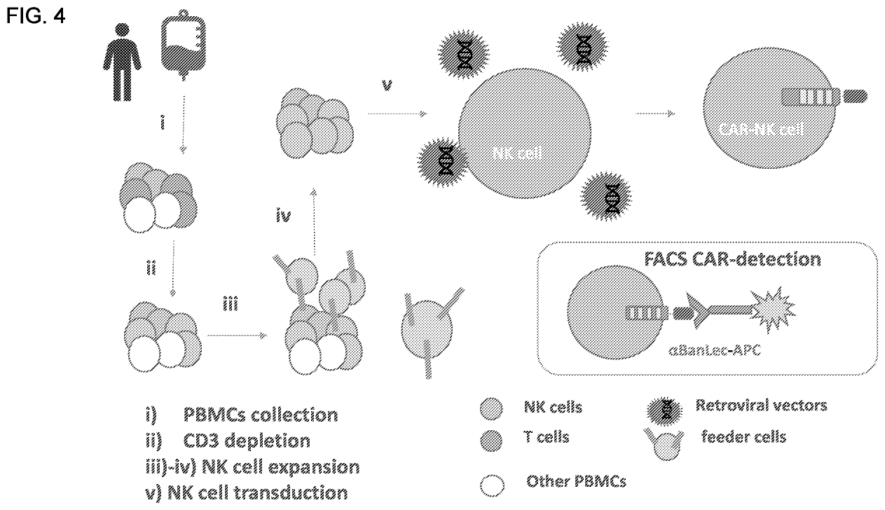

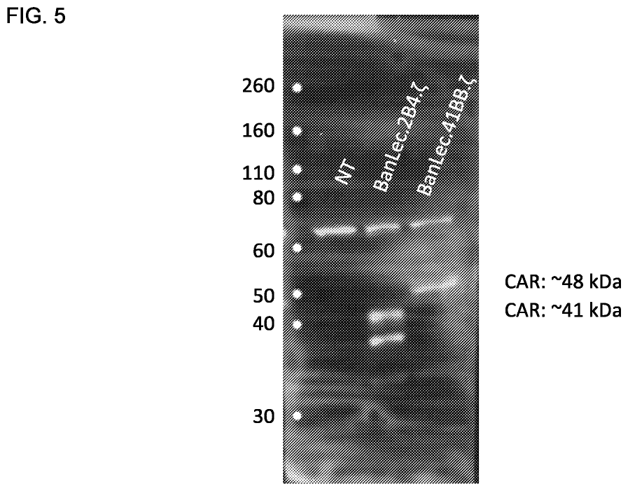

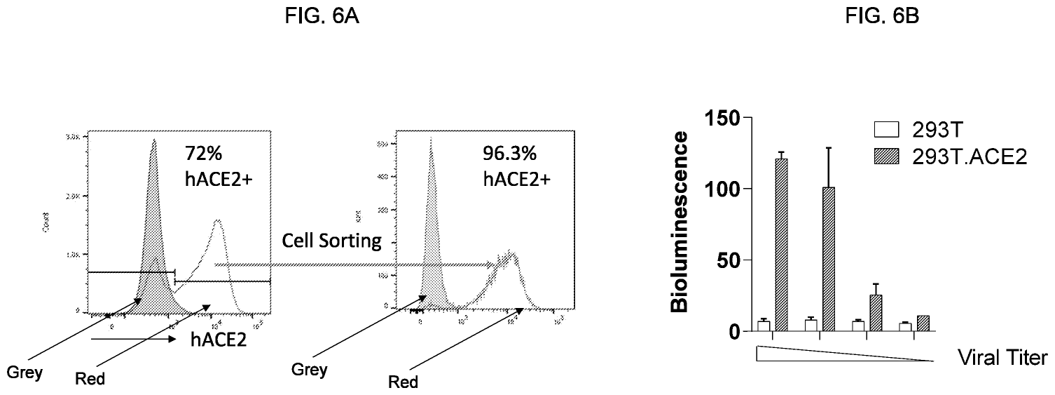

H84T-BanLec CAR was engineered into T cells, exploiting the lectin as an alternate to a conventional single chain variable fragment (scFv) for antigen binding (FIG. 3A). Targeting of glycans is not subject to the antigenic downmodulation seen in clinical trials of targeted cell therapies. The H84T BanLec serves as the extracellular domain of the receptor.

Cancer-specific T cell activation and killing was performed, given the altered glycobiology of transformed cells. Indeed, the H84T-BanLec CAR T cells were functional, with specific T cell activation and anti-cancer killing (FIG. 3B). H84T-BanLec CAR T cells were not toxic to the MRC5 normal lung fibroblast cell line (FIG. 3B). These are a promising therapeutic under development, with patent pending for design and use (see U.S. patent application Ser. No. 16/782,417 which is published as US2020/0247868), incorporated herein by reference in its entirety).

The novel binding mechanism is a first-in-class immunotherapy as prior targeting of glycans has been tested using only antibody-based binding moieties. Glycosite binding via H84T-BanLec as a mechanism of engineered effector cell targeting can be extended to direct cytotoxicity against invasive pathogens decorated with oligomannose-containing glycoproteins. SARS-CoV-2 is one such pathogen.

Oligomannose containing glycan shields surround the SARS-CoV-2 ACE2 receptor binding domain (RBD), which are hypothesized to protect binding topology and are therefore unlikely to be altered by mutagenesis that would otherwise lead to evasion of epitope targeting.

### Example 2: Evaluation of Binding and Antigen Specific Activation of H84T-BanLec CAR NK Cells by SARS-CoV-2 Envelope Proteins

NK cells are engineered with H84T-BanLec.CARs. Recombinant SARS-CoV-2 envelope proteins (S-trimer, M, E) are used to evaluate specific binding and CAR-NK cell activation.

To confirm that the H84T-BanLec binds SARS-CoV-2, commercially sourced recombinant spike envelope proteins, for example, available from Acrobiosystems, Newark, DE (Catalog No: S1N-C5256-100 ug, “SARS-CoV-2 (COVID-19) S1 protein (D614G), His Tag”), are used. These proteins (for example from Acrobiosystems) produces the recombinant proteins in human HEK293T cells to preserve human host derived glycosylation patterns. H84T-BanLec CAR was expressed with 4-1BB and TCRζ signaling domains in T cells FIG. 3A. A CAR with 2B4.ζ intracellular domains can be optimal for NK cell activation.

The existing 2B4.ζ CAR is modified to include the H84T-BanLec extracellular domain (e.g., for targeting viruses). H84T-BanLec CAR NK cells re-generated from healthy donor peripheral blood. Peripheral blood mononuclear cells (PBMCs) are isolated with a density gradient and depleted of CD3 (+) T cells. CD3 (−) PBMCs are stimulated with lethally irradiated K562.41BBL.IL15 feeder cells and maintained in IL-2 (interleukin 2). Moreover, the CARs described herein are NK cells (not T cells), which was confirmed by immunophenotyping.

On day 4 of activation, cells are transduced with replication incompetent retroviruses carrying the coding sequences of the CARs. CAR expression is verified using a monoclonal antibody to H84T-BanLec. Once CAR expression is demonstrated, it will be determined whether H84T-BanLec CAR NK cells bind glycosites present in the of SARS-CoV-2 envelope. His-tagged recombinant envelope proteins (trimeric S—, M-, E-proteins) are used in FACS-based binding assays. Cells are coated with different concentrations of His-envelope proteins and binding is analyzed by flow cytometry. Density of binding will be quantified with Bangs beads (Bangs Laboratories, Inc, Fishers, IN).

### Glycoprotein-Specific Activation of the H84T-BanLec.CAR NK Cells is Evaluated.

Recombinant SARS-CoV-2 envelope proteins are immobilized on tissue culture plates.

H84T-BanLec.CAR NK are added to the plates and sampled at 4, 24, and 48 hrs after activation. NK cells are evaluated by measuring:
Cytokine production (for example, IFNγ (interferon gamma), GM-CSF (Granulocyte-macrophage colony-stimulating factor), TNFα (tumor necrosis factor alpha)),
Expression/upregulation of activating receptors, for example, CD16 (cluster of differentiation 16), NKG2D, CD161 (cluster of differentiation 161), NKp46, or NKp30
Expression/upregulation of activation markers, for example, CD69 (cluster of differentiation 69), or CD94/NKG2C,
Expression/upregulation of inhibitory receptors, for example CD94/NKG2A,
Expression/upregulation of T-cell exhaustion markers such as programmed death ligand 1 (PD-1), and/or
Degranulation of perforin and granzyme.

Additionally, target-specific proliferation of H84T-BanLec.CAR-NK cells is measured. Unmodified NK cells and empty wells are used as controls. Experiments are repeated with at least three independent PBMC donors.

### Example 3: H84T-BanLec. CAR NK Cell Mediated Prevention of SARS-CoV-2 Infection of Human Epithelial Cells

Target cells are engineered to express the ACE2 receptor protein. Engineered cells are coated with SARS-CoV-2 viral glycoproteins and co-cultured with our H84T-BanLec.CAR NK cells. CAR-NKs are evaluated for i.) target-specific proliferation, ii.) cytokine secretion, iii.) immunophenotype, and iv.) target cytotoxicity. Replication incompetent retrovirus pseudotyped with the SARS-COV2 spike protein transduced 293T.hACE2 cell line.

H84T-BanLec.CAR NK cells are a promising therapeutic, capable of clearing circulating virus as well as infected cells. Translation of the H84T-BanLec.CAR NK cells from benchtop to clinic may provide a valuable therapeutic candidate to treat SARS-CoV-2 infection.

Respiratory epithelial cells infected with SARS-CoV-2 retain expression of viral envelope glycoproteins on the epithelial cell membrane. The binding receptor for SARS-CoV-2 is the ACE2 transmembrane protein. HEK293T cells can be engineered to express ACE2 and bind coronaviruses. Cellular models resembling SARS-CoV2 infection are created using recombinant envelope proteins, S-protein pseudotyped lentivirus, and ACE2 engineered HEK293T.

A plasmid encoding myc-tagged human ACE2 cDNA is available from the Addgene plasmid biobank (Addgene, Watertown, MA) and is used for subcloning into a lentiviral expression plasmid. HEK293T cells are transduced with ACE2 containing lentiviral vectors to establish ACE2 surface expression.

Stable introduction of firefly Luciferase (ffLuc) expression using replication incompetent retrovirus sourced from a producer cell line can be used. The ffLuc vector additionally carries GFP. In this way, ACE2.293T.ffLuc.GFP can be generated.

Expression is verified with FACS using hACE2 antibodies. Cells are sorted to purify lines with low, medium, and high ACE2 expression. Once these are purified, hACE2.HEK293T cells are coated with the recombinant trimeric S-protein. S-protein binding to ACE2 on the cell surface is analyzed with FACs to determine the optimal S-protein concentration for receptor saturation.

S-protein coated hACE2.cells are co-cultured with BanLec-CAR NK cells at 10:1, 2:1, 1:2, and 1:10 effector:target cell ratios. NK synapse formation, activation, proliferation, and cytotoxicity is evaluated. NK cell immunophenotype after stimulation is evaluated to determine expression of exhaustion-associated surface markers. Uncoated 293T cell lines and unmodified NK cells are used as controls. All experiments are conducted using at least three independent healthy donor PBMC sources.

Trimeric S-protein binds the ACE2 receptor. H84T-BanLec.CAR NK cells bind S-protein cultured epithelial cells with resultant activation and cytotoxicity. If synapse formation and target killing does not occur, the use of live virus will be done in the assays. Additionally, viral clearance may be tested and H84T-BanLec.CAR NK impact on SARS-CoV2 infectivity.

### Statistical Analysis.

All binding, signaling, activation, and cytotoxicity studies are performed at least in biological triplicate. CAR-NK cells are generated and tested from at least three independent NK cell donors to validate reproducibility. Statistical comparisons between two groups are performed by one-way analysis of variance (ANOVA) with Tukey post-tests. Two-way ANOVA corrected for comparison using the method of Sidek are used for comparison of 3 or more groups. Calculated differences of p<0.05 are considered statistically significant.

### REFERENCES

1. Koshte, V. L., et al., Isolation and characterization of BanLec-I, a mannoside-binding lectin from Musa paradisiac (banana). Biochem J, 1990. 272 (3): p. 721-6.
2. Hopper, J. T. S., et al., The Tetrameric Plant Lectin BanLec Neutralizes HIV through Bidentate Binding to Specific Viral Glycans. Structure, 2017. 25 (5): p. 773-782 e5.
3. Li, W., et al., Angiotensin-converting enzyme 2 is a functional receptor for the SARS coronavirus. Nature, 2003. 426 (6965): p. 450-4.
4. Wrapp, D., et al., Cryo-EM structure of the 2019-nCOV spike in the prefusion conformation. Science, 2020. 367 (6483): p. 1260-1263.
5. Watanabe, Y., et al., Site-specific analysis of the SARS-CoV-2 glycan shield. BioRxiv, 2020
6. Zhang, Y., et al., Site-specific N-glycosylation Characterization of Recombinant SARS-CoV-2 Spike Proteins using High-Resolution Mass Spectrometry. bioRxiv, 2020.
7. Shajahan, A., et al., Deducing the N-and O-glycosylation profile of the spike protein of novel coronavirus SARS-CoV-2. bioRxiv, 2020.
8. Coves-Datson, E. M., et al., A molecularly engineered antiviral banana lectin inhibits fusion and is efficacious against influenza virus infection in vivo. Proc Natl Acad Sci USA, 2020. 117 (4): p. 2122-2132.
9. Swanson, M. D., et al., A lectin isolated from bananas is a potent inhibitor of HIV replication. J Biol Chem, 2010. 285 (12): p. 8646-55.
10. Mitchell, C. A., K. Ramessar, and B. R. O'Keefe, Antiviral lectins: Selective inhibitors of viral entry. Antiviral Res, 2017. 142: p. 37-54.
11. Swanson, M. D., et al., Engineering a therapeutic lectin by uncoupling mitogenicity from antiviral activity. Cell, 2015. 163 (3): p. 746-58.
12. Imai, C., S. Iwamoto, and D. Campana, Genetic modification of primary natural killer cells overcomes inhibitory signals and induces specific killing of leukemic cells. Blood, 2005. 106 (1): p. 376-83.
13. Liu, E., et al., Use of CAR-Transduced Natural Killer Cells in CD19-Positive Lymphoid Tumors. N Engl J Med, 2020. 382 (6): p. 545-553.
14. Bonifant, C. L., et al., Toxicity and management in CAR T-cell therapy. Mol Ther Oncolytics, 2016. 3: p. 16011.
15. Bonifant, C. L., et al., CD123-Engager T Cells as a Novel Immunotherapeutic for Acute Myeloid Leukemia. Mol Ther, 2016. 24 (9): p. 1615-26.
16. Krawczyk, E., et al., T-cell Activity against AML Improved by Dual-Targeted T Cells Stimulated through T-cell and IL7 Receptors. Cancer Immunol Res, 2019. 7 (4): p. 683-692.
17. Zolov, S. N., S. P. Rietberg, and C. L. Bonifant, Programmed cell death protein 1 activation preferentially inhibits CD28.CAR-T cells. Cytotherapy, 2018. 20 (10): p. 1259-1266.
18. Giamarellos-Bourboulis, E. J., et al., Complex Immune Dysregulation in COVID-19 Patients with Severe Respiratory Failure. Cell Host Microbe, 2020.
19. Wang, F., et al., Characteristics of peripheral lymphocyte subset alteration in COVID-19 pneumonia. J Infect Dis, 2020.
20. Qin, C., et al., Dysregulation of immune response in patients with COVID-19 in Wuhan. China. Clin Infect Dis, 2020.
21. Siegler, E. L., et al., Off-the-Shelf CAR-NK Cells for Cancer Immunotherapy. Cell Stem Cell, 2018. 23 (2): p. 160-161.
22. Steentoft, C., et al., Glycan-directed CAR-T cells. Glycobiology, 2018. 28 (9): p. 656-669.
23. Procko, E., The sequence of human ACE2 is suboptimal for binding the S spike protein of SARS coronavirus 2. bioRxiv, 2020.
24. Fujisaki, H., et al., Expansion of highly cytotoxic human natural killer cells for cancer cell therapy. Cancer Res, 2009. 69 (9): p. 4010-7.
25. Bonifant, C. L. and S. K. Tasian, The future of cellular immunotherapy for childhood leukemia. Curr Opin Pediatr, 2020. 32 (1): p. 13-25.

### Example 4

### Materials and Methods

### Cell Lines

HEK293T cells were purchased from the American Type Culture Collection (ATCC, Manassas, VA) and grown in Dulbecco's Modified Eagle Medium (DMEM; ThermoFisher Scientific Waltham, MA), supplemented with 10% Fetal Bovine Serum (FBS; HyClone, Logan, UT). High-expressing human ACE2 (hACE2) 293T cells were created by first subcloning hACE2 (pCEP4-myc-ACE21 was a gift from Erik Procko: Addgene plasmid #141185; http://n2t.net/addgene: 141185; RRID: Addgene_141185, Addgene, Watertown, MA) into a pCDH lentiviral backbone (System Biosciences, Palo Alto, CA). Vesicular stomatitis virus G glycoprotein (VSV-G) Pseudotyped HIV-derived lentiviral particles were then produced using the pPACKHI HIV Lentivector Packaging Kit (System Biosciences, Palo Alto, CA) according to the manufacturer's instructions and used for 293T.hACE2 cell generation. Cells with high ACE2 expression, as validated with flow cytometry, were then isolated via fluorescence-activated cell sorting (FACS). hACE2.293T cells used for cytotoxicity analysis were additionally modified with retroviral vectors to express an enhanced green fluorescent protein (GFP) firefly luciferase fusion gene (GFP.ffLuc).⁵³ GFP-positive cells were sorted and maintained in the appropriate complete growth medium. GFP expression was confirmed through flow cytometric analysis and luciferase expression was confirmed using D-luciferin and quantification of bioluminescence. All cells were maintained in a humidified atmosphere containing 5% CO2 at 37° C.

### Chimeric Antigen Receptor (CAR) Synthesis

The sequence of H84T-BanLec³² was synthesized (GeneArt, ThermoFisher Scientific) and subcloned into a pSFG⁵⁴ retroviral vector backbone linked to the intracellular domains of 4-1BB (CD137) and TCRζ³⁶ More specifically, the lectin is linked to a CD8α hinge and transmembrane domain, CD137 intracellular domain, and the intracellular domain of TCRζ (H84T-BanLec.4-1BB.ζ). Transgenic sequence fidelity was validated by Sanger sequencing (Johns Hopkins Genetic Resources Core Facility).

### Generation of CAR-NK Cells

Peripheral blood mononuclear cells were isolated from healthy donor leukopaks (Anne Arundel Medical Blood Donor Center, Annapolis, MD). T cells were then depleted with CD3-microbeads (Militenyi Biotec, Cologne, Germany). CD3+ cell depletion was verified with flow cytometry using Phycoerythrin (PE)-conjugated anti CD3 (clone: HIT3a, BD Biosciences, Franklin Lakes, NJ) and Brilliant Violet (BV) 421-conjugated anti-CD56 (clone: HCD56, BioLegend, San Diego, CA) antibodies. CD3-depleted peripheral blood mononuclear cells were stimulated on day 0 with lethally irradiated K562 feeder cells expressing membrane bound IL15 and 4-1BB ligand⁴⁵ at a 1:1 ratio. Cells were maintained in SGCM media (CellGenix, Freiburg, Germany) with 10% FBS and 2 mMol glutaMAX (ThermoFisher) supplemented with recombinant human interleukin (IL)-2 (200 IU/mL, preclinical biorepository, National Cancer Institute). NK cell transduction was performed on day 4 of the culture using transiently produced replication incompetent RD114-pseudotyped retroviral particles immobilized on RetroNectin (Clontech Laborotories, Palo Alto, CA).

### Determination of Vector Copy Number (VCN)

Primer/probe-FAM was designed to the MMLV-derived psi present in pSFG⁵⁴ and purchased from ThermoFisher Scientific. RNAseP primer/probe-VIC/TAMRA mix (Applied Biosystems #4403326) was used as comparison. Genomic DNA was isolated from CAR-NK cells and 25 ng used for amplification with TaqMan Universal PCR Mastermix (ThermoFisher) and the above primer/probe mixes on a C1000 Touch Thermal Cycler (Bio-Rad, Hercules, CA). The following amplification conditions were used: 50° C. for 2 minutes, 95° C. for 10 minutes, 40 cycles of 95° C. for 15 seconds, 60° C. for 1 minute. No-template, unmodified NK cells and a condition containing only plasmid were used as controls. Vector copy number calculation was performed using the 2^{-ΔCt} method. 55

### SARS-CoV-2 Spike Pseudotyped Viral Assays

SARS-CoV-2 S-protein pseudotyped replication incompetent lentiviral particles were produced by first transfecting 293T with GeneJuice transfection reagent (Millipore-Sigma) and SARS-Related Coronavirus 2, Wuhan-Hu-1 Spike-Pseudotyped Lentiviral Kit (The following reagent was obtained through BEI Resources, NIAID, NIH: SARS-Related Coronavirus 2, Wuhan-Hu-1 Spike-Pseudotyped Lentiviral Kit, NR-52948: individual plasmids+0.56 at indicated ratios (Supplemental Table 1); BEI Resources Repository, Manassas, VA). S-protein pseudotyped viral supernatant was collected 48 h after transfection. 1.25×10⁴ hACE2.293T cells were plated on day-1 in black 96-well microplates (Corning, Corning, NY). Parental 293T cells served as a control for nonspecific cell transduction. On Day 0, S-pseudoviral titrations (1:1, 1:5, 1:25, 1:125) were added and the plate was centrifuged at 800 g for 30′ at 32° C. Cells were then incubated at 37° C. in 5% CO₂. At 48 h post-transduction, the viral-containing supernatant was aspirated and 150 ug/ml D-Luciferin containing fresh media added. BLI was measured and reactive light units (RLU) determined after subtraction of virus-only background. For co-culture assays containing NK cells and pseudovirus transduced target cells, pseudoviral particles were titrated first on hACE2.293T cells and added to achieve 100-200 maximum RLU. NK cells were added to the hACE2.293T and pseudovirus immediately after centrifugation. Decreased infectivity was calculated as 100*(baseline BLI−co-culture BLI)/(baseline BLI−background). Baseline BLI was measured from wells containing only hACE2.293T and pseudovirus. The assay was performed in experimental triplicate per donor.

### Flow Cytometry

ACE2 expression on 293T cells was validated with flow cytometric analysis, utilizing staining with Alexa Fluor 647 conjugated anti-ACE2 Ab (Clone #535919; R&D Systems, Minneapolis, MT). CAR expression on the surface of transduced NK cells was evaluated 4 and 14 days post-transduction using primary staining with H84T. BanLec Ab 33 and secondary staining with AlexaFluor647-anti-rabbit F (ab) 2 (Jackson ImmunoResearch, West Grove, PA). All samples were acquired on FACSCelesta Cell Analyzer (BD) and analyzed with FlowJo software (v10.6.1). Cell sorting was performed on FACSMelody (BD).

### Binding of Spike Protein to 293T.ACE2

Histidine (His)-tagged recombinant S-proteins: trimera (SPN—C52H9) and D614G trimera (SPN—C52H3) were purchased from ACROBiosystems (Newark, DE). A His-tagged receptor binding domain (RBD) was purchased from R&D Systems (10-500-CV-100, Minneapolis, MN). 293T, hACE2.293T, NK cells, and H84T-BanLec CAR-NK cells were coated with 50 ng of recombinant protein, then stained first with His antibody (R&D Systems), followed by PE-anti-mouse F (ab) 2 (R&D Systems) and analyzed using flow cytometry.

### Western Blot

NK cells were lysed in RIPA lysis buffer with protease (complete) and phosphatase (PhosSTOP) inhibitor cocktails (Sigma-Aldrich, St. Louis, MO) on ice. Protein quantification was performed using Pierce BCA protein assay kit (cat #23228 and #23224, ThermoFisher) and iMark plate reader (Bio-Rad). Electrophoresis was conducted using Novex WedgeWell 10% Bis-Tris Mini Gels (Thermo Fisher) and protein transferred to polyvinylidene difluoride (PVDF) membrane. Western blot analysis was performed with the following antibodies: mouse anti-human CD247 (clone 1D4; BD Biosciences), mouse anti-human phosphory lated CD247 (pY142, clone K25-407.69; BD Biosciences), and rabbit anti-human H84T. BanLec Ab.³³ Membranes were stripped with Restore Western Blot Stripping Buffer (ThermoFisher) and used again for analysis with polyclonal rabbit anti-human glyceraldehyde 3-phosphate dehydrogenase (GAPDH) (Novus Biologicals, Littleton, CO).

### Cytokine Secretion

NK cells were cultured with SARS-CoV-2-pseudovirus transduced hACE2.293T at a 1:1 ratio. Following 48 h of culture, supernatant was harvested and analyzed for interferon (IFN)γ or tumor necrosis factor (TNF)α using enzyme-linked immunosorbent assay (ELISA) kits (Quantikine; R&D Systems) according to the manufacturer's instruction. Conditions without pseudoviral particles were included as controls.

### Cytotoxicity Assay

NK cells were co-cultured for 48 h with hACE2.293T.ffLuc at the indicated effector:target (E/T) ratios. D-luciferin was added to plate and BLI measured per well. Mean percentage of specific lysis of triplicate samples was calculated as 100*(spontaneous death−experimental death)/(spontaneous death−background). Spontaneous death was measured with control wells containing only target cells.

### Statistical Analysis

All analyses were performed using GraphPad Prism Software (v9). For comparisons of 2 groups, unpaired t tests with Welch correction were used.

The following Table 1 lists all the plasmids of SARS-Related Coronavirus 2, Wuhan-Hu-1 Spike-Pseudotyped Lentiviral Kit that were used for generation of S-protein pseudotyped lentiviral particles. Plasmid name, type, inserted gene and catalog numbers as well as the microgram ratio used for transfection are listed.

TABLE 1
Plasmids in SARS-Related Coronavirus 2, Wuhan-Hu-1 Spike-Pseudotyped Lentiviral Kit

| | | | BEI Resources | |
|---|---|---|---|---|
| Plasmid Name | Plasmid Type | Insert | Catalog Number | Microgram-ratio* |
| pHDM.CMV.Spike | Viral Entry Protein | Spike (S) | NR-52514 | 1.7 |
| | | glycoprotein | | |
| pHAGE.CMV.ffLuc.IRES.ZSG | Lentiviral Backbone | Firefly Luciferase; | NR-52516 | 5 |
| | | ZsGreen | | |
| pHDM.HIV gag-pol | Helper Plasmid | Gag; pol | NR-52517 | 1.1 |
| pHDM.HIV HIV tat1b | Helper Plasmid | Tat1b | NR-52518 | 1.1 |
| pRC.CMV.HIV rev1b | Helper Plasmid | Rev1b | NR-52519 | 1.1 |

*The ratio of micrograms of each plasmid used during transfection of 293T cells

### Results

### H84T-BanLec.4-1BB.ζ CAR is Stably Expressed in Human NK Cells

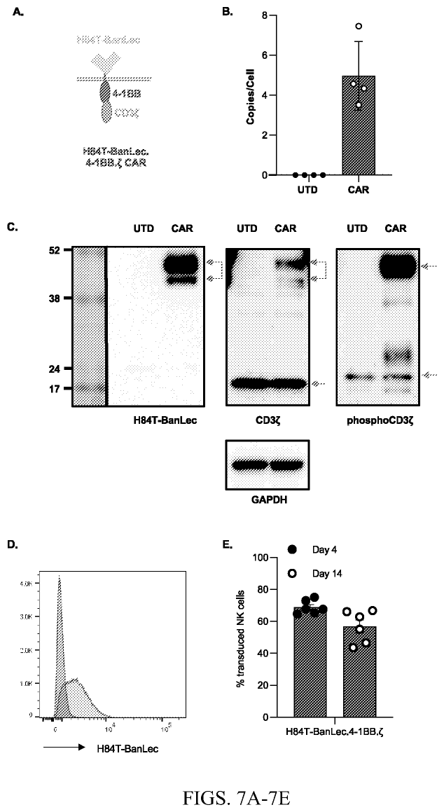

We synthesized the H84T-banana lectin (H84T-BanLec) 32 sequence and subcloned this in place of the extracellular binding domain of an existing 4-1BB.ζ CAR.³⁶ Our complete CAR was comprised of H84T-BanLec, CD8a hinge and transmembrane domains, and the intracellular domains of 4-1BB and the CD35 chain (FIG. 7A). We produced replication incompetent retrovirus carrying our CAR sequence and used this to generate H84T-BanLec CAR NK cells. We measured a median of 4.5 integrated CAR copies per cell (range 3.5-7.45, FIG. 7B) and verified expression of CAR protein using Western blot (FIG. 7C). We observed constitutive CD35 phosphorylation of the CAR in our transduced cells, a finding that has previously been associated with CAR tonic signaling (FIG. 7C).37.38 We verified surface expression using flow cytometry and measured stable expression of the H84T-BanLec.4-1BB.ζ CAR on the surface of human NK cells (day 4 post-transduction: median [range], 67.5% CAR-positive [64.7-75%], day 14 post-transduction: 58.9% CAR-positive [43.6-66.7%], FIG. 7D, 7E).

hACE2-Expressing 293T Cells Bind SARS-CoV-2 Envelope Proteins

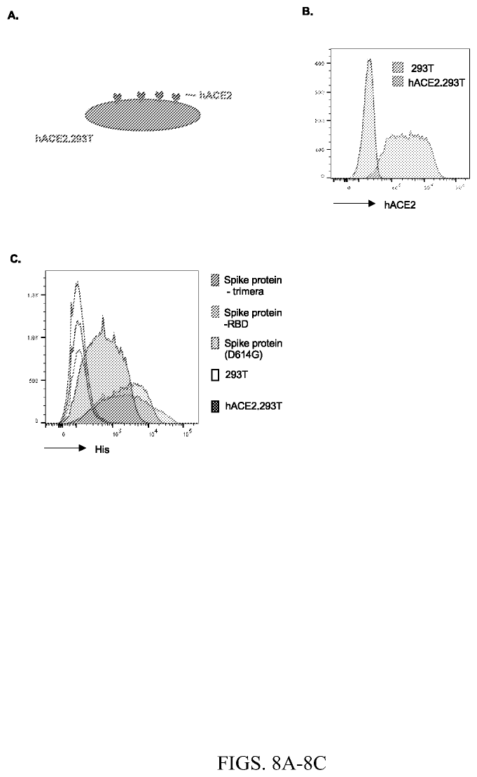

In order to model SARS-CoV-2 infection, we engineered 293T cells to constitutively express the ACE2 transmembrane protein (FIG. 8A, 8B). Human ACE2 is the binding partner for SARS-CoV-2.^{24,39} We found that our hACE2.293T cells bound trimeric S-protein, the S-protein Receptor Binding Domain (RBD), and the D614G mutated²¹ S-protein (FIG. 8C). Unmodified 293T cells did not bind S-proteins (FIG. 8C).

### H84T-BanLec CAR-NK Cells Decrease Infectivity of Pseudoviral Particles

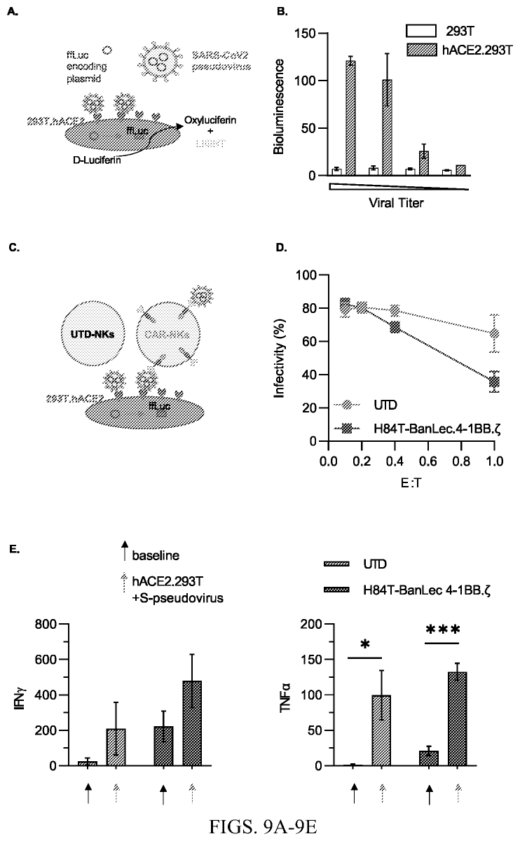

We used SARS-CoV-2 envelope pseudotyping of a replication deficient lentiviral vector⁴⁰ in order to evaluate whether H84T-BanLec CAR NK cells could reduce SARS-CoV-2 infectivity. We first tested hACE2.293T transduction using S-protein pseudotyped lentiviral particles. The pseudotyped vector carried firefly luciferase (ffLuc, FIG. 9A). Viral entry into cells was verified by quantification of bioluminescence (BL) emission following addition of D-Luciferin to virally transduced cells. Functionality of our assay was confirmed with observed BL emission of transduced hACE2.293T cells at all tested viral titrations. In contrast, 293T without ACE2 expression were not transduced, confirming specificity of viral binding and entry dependent on hACE2 (FIG. 9B).

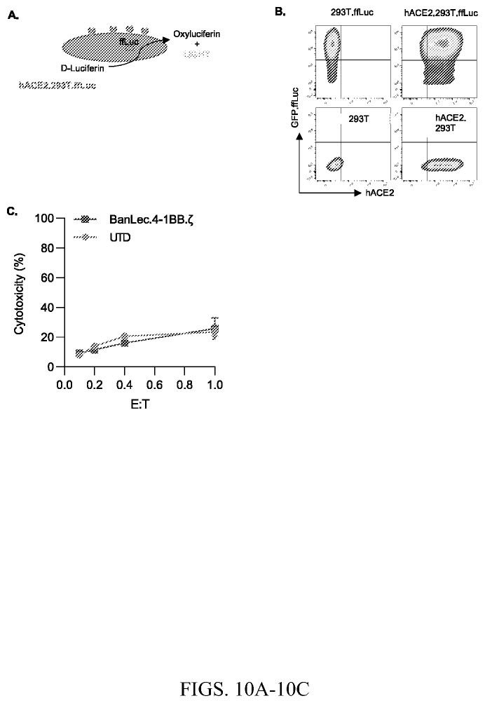

We next investigated whether H84T-BanLec CAR-NK cells could decrease S-protein mediated hACE2.293T transduction. NK cells (CAR-NKs or unmodified) were plated with hACE2.293T and freely circulating pseudoviral particles (FIG. 9C). We observed a reduction in SARS-CoV-2 pseudoviral infectivity of hACE2.293T cells when H84T-BanLec CAR-NK cells were present (FIG. 9D). Viral infectivity was reduced by both unmodified and H84T-CAR NK cells, but was more pronounced when CAR-NK cells were present (mean % pseudovirus infectivity+/−SEM of hACE2.293T in cocultures with unmodified NK vs. H84T-BanLec CAR-NK: 65+/−11% vs 35%+/−6% for 1:1 effector-to-target ratio, p=0.05:78+/−3% vs 68%+/−3% for 1:2.5 effector-to-target ratio, p=0.03; FIG. 9D). NK cells are reactive immune effector cells with cytotoxic potential against allogeneic targets. Therefore, we analyzed the killing capacity of NK cells against hACE2.293T cells to determine whether differences seen in infectivity could be explained by nonspecific target killing. There were no observed differences between CAR- and unmodified NK cell killing of hACE2.293T cells across a range of effector-to-target (E:T) ratios (mean % cytotoxicity+/−SEM of unmodified NK vs. H84T-BanLec CAR-NK: 23.5+/−2.7% vs. 25.9+/−7% in 1:1 E:T ratio, p=0.76:20.6+/−2% vs 16.1%+/−1% in 1:2.5 E:T ratio, p=0.1, see FIG. 10A-C). Moreover, the measured cytotoxicity of NK cells against 293T cells was roughly equivalent to the decrease in infectivity noted with unmodified NK cells, suggesting background cytotoxicity may have contributed to decreased viral entry in the absence of H84T-BanLec viral binding.

### H84T-BanLec CAR-NK Cells are Strongly Activated by Virus

We evaluated the activation of NK cells in our pseudovirus assay. Both unmodified and H84T-BanLec CAR-NK cells were stimulated to secrete inflammatory mediators when co-cultured with pseudoviral particles and virally infected cells, including IFNγ (mean pg/ml+/−SEM of NK cells at baseline vs. in coculture with hACE2.293T and S-pseudovirus; unmodified NK: 24+/−11.6 vs. 209.4+/−86.1, p=0.16; CAR-NK: 221.9+/−49.7 vs. 479.2+/−86.7, p=0.07: n=3, FIG. 9E) and TNFα (unmodified NK: 1.2+/−0.6 vs. 99.3+/−20.1, p<0.05; CAR-NK: 20.9+/−3.6 vs. 132.6+/−6.8, p<0.001; n=3, FIG. 3E) CAR-NK cells showed overall higher cytokine secretion both at baseline and with viral stimulation.

### Discussion

We have demonstrated successful generation of CAR-NK cells expressing extracellular H84T-BanLec linked to intracellular activation domains. Surface expression of the lectin-containing CAR was associated with tonic phosphorylation of CAR-CD35, validating its molecular functionality. We also engineered 293T cells to express ACE2, the SARS-CoV-2 receptor protein. We employed a model of SARS-CoV-2 infection that used a lentiviral vector pseudotyped with the S-protein. Virus infectivity was inhibited by H84T-BanLec CAR-NK cells. Moreover, our CAR-NK cells increased their secretion of IFNγ and TNFα after encountering virally infected cells. Increased inflammatory cytokine secretion was also observed to a lesser extent in unmodified NK cells, illustrating NK cell innate anti-viral potency.

CAR-T and CAR-NK cells are emerging immunotherapies with great promise. Typically, the expressed synthetic receptors are designed to bind surface protein. However, in our study, we designed a CAR making use of a unique extracellular moiety with binding properties dependent on target glycosylation. Targeting of glycoprotein, and specifically N-glycosylation products, with a CAR is rare. We believe that we describe the first CAR targeting the N-glycans dispersed on SARS-CoV-2 envelope proteins. Furthermore, to our knowledge this is the first lectin-based CAR designed and functionally tested in pre-clinical study. As lectins have evolved over millions of years to be highly potent and selective, the H84T-BanLec CAR represents an entirely new approach in that it targets aberrant glycosylation patterns in viral proteins. This methodology has the potential to be applied against cancer and other target cells as well.

During viral infections NK cells have a central role as first responders.¹⁸ Viral infections can activate NK cells to produce IFNγ, TNFα, and other immunity-enhancing mediators that prime the adaptive immune response. 13.18.41 However, many viruses, including SARS-CoV-2, deploy strategies to evade NK cell surveillance.^{42,43} CAR expression on the surface of NK cells can potentiate endogenous and antigen-specific activation and target killing.⁴⁴ This boosted function may render CAR-NK cells superior effectors in clearing circulating virus and virally infected cells. Appropriate antigen targeting and a precise combination of intracellular signaling domains are critical to direct CAR-NK cell behavior. We and others⁴⁴⁻⁴⁷ have found 4-1BB and CD3ζ intracellular domains to be a good combination for NK cell activation. The vast majority of CAR constructs contain an extracellular single chain variable fragment derived from a monoclonal antibody for protein binding.²³ Instead, we used a lectin with specific binding to high mannose, an altered glycosylation pattern common to viral envelopes.^{26,27,29,30,48} Our glycoprotein targeting mitigates the potential risk of antigen downregulation, a mechanism commonly employed by cancer cells to evade targeted immunotherapies.⁴⁹ CAR binding to envelope glycoproteins has the potential to not only neutralize and eliminate circulating virus, but also to clear infected cells with retained envelope proteins on their surface.

We observed decreased infectivity likely secondary to specific viral clearance by our CAR-NK cells. We also observed stable CAR.ζ phosphorylation and heightened activation of CAR-NK cells when compared to unmodified NK cells. This 4-1BB.ζ induced activation may prevent the dysfunction seen in circulating NK cells of patients with COVID-19.^{11.50} similar to 4-1BB.ζ CAR mitigation of T cell exhaustion.³⁸ Ultimately, investigation of H84T-BanLec CAR-NK cell efficacy against wild-typeSARS-CoV-2 virus and in animal models of SARS-CoV-2 infection is needed.

Acute deterioration with COVID-19 requires emergency treatment options available at the ready. In comparison to T cells, which must be manufactured from autologous cells in order to prevent graft-versus-host disease, NK cells can be infused from allogeneic donors without this risk.^{51,52} Aliquots of manufactured CAR-NK cells can be frozen and stored to establish a master cell bank capable of treating patients suffering from COVID-19 and in need of adequate immune function. Indeed, there are open clinical trials investigating the potential role of NK cell (NCT04280224, NCT04365101, NCT04634370, NCT04797975, NCT04900454) and CAR-NK cell (NCT04324996) adoptive transfer as COVID-19 treatments. Our findings support the hypothesis that CAR-NK cells expressing H84T-BanLec can mediate SARS-CoV-2 viral clearance. Taken together with the favorable innate antiviral characteristics and capability for allogeneic infusion of NK cells, H84T-BanLec CAR-NK cells may have promise as an effective cellular therapy against SARS-CoV-2 infection. Translation to the clinic could potentially impact days of hospitalization and survival rates of COVID-19 patients.

### References: Example 4

1. Mohamadian, M., Chiti, H., Shoghli, A., Biglari, S., Parsamanesh, N., and Esmaeilzadeh, A. (2021). COVID-19: Virology, biology and novel laboratory diagnosis. J Gene Med 23, e3303. 10.1002/jgm.3303.
2. Baden, L. R., El Sahly, H. M., Essink, B., Kotloff, K., Frey, S., Novak, R., Diemert, D., Spector, S. A., Rouphael, N., Creech, C. B., McGettigan, J., et al. (2021). Efficacy and Safety of the mRNA-1273 SARS-CoV-2 Vaccine. N Engl J Med 384, 403-416. 10.1056/NEJMoa2035389.
3. Polack, F. P., Thomas, S., Kitchin, N., Absalon, J., Gurtman, A., Lockhart, S., Perez, J. L., Perez Marc, G., Moreira, E. D., Zerbini, C., Bailey, R., et al. (2020). Safety and Efficacy of the BNT162b2 mRNA Covid-19 Vaccine. N Engl J Med 383, 2603-2615. 10.1056/NEJMoa2034577.
4. Giamarellos-Bourboulis, E. J., Netea, M. G., Rovina, N., Akinosoglou, K., Antoniadou, A., Antonakos, N., Damoraki, G., Gkavogianni, T., Adami, M. E., Katsaounou, P., Ntaganou, M., et al. (2020). Complex Immune Dysregulation in COVID-19 Patients with Severe Respiratory Failure. Cell Host Microbe. 10.1016/j.chom.2020.04.009.
5. Wang, F., Nie, J., Wang, H., Zhao, Q., Xiong, Y., Deng, L., Song, S., Ma, Z., Mo, P., and Zhang, Y. (2020). Characteristics of peripheral lymphocyte subset alteration in COVID-19 pneumonia. J Infect Dis. 10.1093/infdis/jiaa150.
6. Qin, C., Zhou, L., Hu, Z., Zhang, S., Yang, S., Tao, Y., Xie, C., Ma, K., Shang, K., Wang, W., and Tian, D. S. (2020). Dysregulation of immune response in patients with COVID-19 in Wuhan, China. Clin Infect Dis. 10.1093/cid/ciaa248.
7. Files, J. K., Boppana, S., Perez, M. D., Sarkar, S., Lowman, K. E., Qin, K., Sterrett, S., Carlin, E., Bansal, A., Sabbaj, S., Long, D. M., et al. (2021). Sustained cellular immune dysregulation in individuals recovering from SARS-CoV-2 infection. J Clin Invest 131. 10.1172/JCI140491.
8. Cizmecioglu, A., Akay Cizmecioglu, H., Goktepe, M. H., Emsen, A., Korkmaz, C., Esenkaya Tasbent, F., Colkesen, F., and Artac, H. (2021). Apoptosis-induced T-cell lymphopenia is related to COVID-19 severity. J Med Virol 93, 2867-2874. 10.1002/jmv.26742.
9. Diao, B., Wang, C., Tan, Y., Chen, X., Liu, Y., Ning, L., Chen, L., Li. M., Liu, Y., Wang, G., Yuan, Z., et al. (2020). Reduction and Functional Exhaustion of T Cells in Patients With Coronavirus Disease 2019 (COVID-19). Front Immunol/1, 827. 10.3389/fimmu.2020.00827.
10. Liu, J., Li, S., Liu, J., Liang, B., Wang, X., Wang, H., Li, W., Tong, Q., Yi, J., Zhao, L., Xiong, L., et al. (2020). Longitudinal characteristics of lymphocyte responses and cytokine profiles in the peripheral blood of SARS-CoV-2 infected patients. EBioMedicine 55, 102763. 10.1016/j.ebiom.2020.102763.
11. Wilk, A. J., Rustagi, A., Zhao, N. Q., Roque, J., Martinez-Colon, G. J., McKechnie, J. L., Ivison, G. T., Ranganath, T., Vergara, R., Hollis, T., Simpson, L. J., et al. (2020). A single-cell atlas of the peripheral immune response in patients with severe COVID-19. Nat Med 26, 1070-1076. 10.1038/s41591-020-0944-y.
12. Hammer, Q., Ruckert, T., and Romagnani, C. (2018). Natural killer cell specificity for viral infections. Nat Immunol 19, 800-808. 10.1038/s41590-018-0163-6.
13. Lam, V. C., and Lanier, L. L. (2017). NK cells in host responses to viral infections. Curr Opin Immunol 44, 43-51. 10.1016/j.coi.2016.11.003.
14. Martinet, L., and Smyth, M. J. (2015). Balancing natural killer cell activation through paired receptors. Nat Rev Immunol 15, 243-254. 10.1038/nri3799.
15. Prager, I., and Watzl, C. (2019). Mechanisms of natural killer cell-mediated cellular cytotoxicity. J Leukoc Biol 105, 1319-1329. 10.1002/JLB.MR0718-269R.
16. Koutsakos, M., McWilliam, H. E. G., Aktepe, T. E., Fritzlar, S., Illing, P. T., Mifsud, N. A., Purcell, A. W., Rockman, S., Reading, P. C., Vivian, J. P., Rossjohn, J., et al. (2019). Downregulation of MHC Class I Expression by Influenza A and B Viruses. Front Immunol 10, 1158. 10.3389/fimmu.2019.01158.
17. Lodoen, M. B., and Lanier, L. L. (2005). Viral modulation of NK cell immunity. Nat Rev Microbiol 3, 59-69. 10.1038/nrmicro1066.
18. Brandstadter, J. D., and Yang, Y. (2011). Natural killer cell responses to viral infection. J Innate Immun 3, 274-279. 10.1159/000324176.
19. Ma, M., Badeti, S., Chen, C. H., Pinter, A., Jiang, Q., Shi, L., Zhou, R., Xu, H., Li, Q., Gause, W., and Liu, D. (2021). CAR-NK Cells Effectively Target the D614 and G614 SARS-CoV-2-infected Cells. bioRxiv. 10.1101/2021.01.14.426742.
20. Wei, J., Han, X., Bo, J., and Han, W. (2019). Target selection for CAR-T therapy. J Hematol Oncol 12, 62. 10.1186/s13045-019-0758-x.
21. Li, Q., Wu, J., Nie, J., Zhang, L., Hao, H., Liu, S., Zhao, C., Zhang, Q., Liu, H., Nie, L., Qin, H., et al. (2020). The Impact of Mutations in SARS-CoV-2 Spike on Viral Infectivity and Antigenicity. Cell 182, 1284-1294 e1289. 10.1016/j.cell.2020.07.012.
22. Harvey, W. T., Carabelli, A. M., Jackson, B., Gupta, R. K., Thomson, E. C., Harrison, E. M., Ludden, C., Reeve, R., Rambaut, A., Consortium, C.-G. U., Peacock, S. J., et al. (2021). SARS-CoV-2 variants, spike mutations and immune escape. Nat Rev Microbiol 19, 409-424. 10.1038/s41579-021-00573-0.
23. Guedan, S., Calderon, H., Posey, A. D., Jr., and Maus, M. V. (2019). Engineering and Design of Chimeric Antigen Receptors. Mol Ther Methods Clin Dev 12, 145-156. 10.1016/j.omtm.2018.12.009.
24. Li, W., Moore, M. J., Vasilieva, N., Sui, J., Wong, S. K., Berne, M. A., Somasundaran, M., Sullivan, J. L., Luzuriaga, K., Greenough, T. C., Choe, H., et al. (2003). Angiotensin-converting enzyme 2 is a functional receptor for the SARS coronavirus. Nature 426, 450-454. 10.1038/nature02145.
25. Wrapp, D., Wang, N., Corbett, K. S., Goldsmith, J. A., Hsieh, C. L., Abiona, O., Graham, B. S., and Mclellan, J. S. (2020). Cryo-EM structure of the 2019-nCOV spike in the prefusion conformation. Science 367, 1260-1263. 10.1126/science.abb2507.
26. Watanabe, Y., Allen, J. D., Wrapp, D., Mclellan, J. S., and Crispin, M. (2020). Site-specific analysis of the SARS-CoV-2 glycan shield. BioRxiv.
27. Zhang, Y., Zhao, W., Mao, Y., Wang, S., Zhong, Y., Su, T., Gong, M., Lu, X., Cheng, J., and Yang, H. (2020). Site-specific N-glycosylation Characterization of Recombinant SARS-CoV-2 Spike Proteins using High-Resolution Mass Spectrometry. bioRxiv.
28. Shajahan, A., Supekar, N. T., Gleinich, A. S., and Azadi, P. (2020). Deducing the N- and O-glycosylation profile of the spike protein of novel coronavirus SARS-CoV-2. bioRxiv.
29. Koshte, V. L., van Dijk, W., van der Stelt, M. E., and Aalberse, R. C. (1990). Isolation and characterization of BanLec-I, a mannoside-binding lectin from Musa paradisiac (banana). Biochem J 272, 721-726. 10.1042/bj2720721.
30. Hopper, J. T. S., Ambrose, S., Grant, O. C., Krumm, S. A., Allison, T. M., Degiacomi, M. T., Tully, M. D., Pritchard, L. K., Ozorowski, G., Ward, A. B., Crispin, M., et al. (2017). The Tetrameric Plant Lectin BanLec Neutralizes HIV through Bidentate to Viral Binding Specific Glycans. Structure 25, 773-782 e775. 10.1016/j.str.2017.03.015.
31. Swanson, M. D., Winter, H. C., Goldstein, I. J., and Markovitz, D. M. (2010). A lectin isolated from bananas is a potent inhibitor of HIV replication. J Biol Chem 285, 8646-8655. 10.1074/jbc.M109.034926.
32. Swanson, M. D., Boudreaux, D. M., Salmon, L., Chugh, J., Winter, H. C., Meagher, J. L., Andre, S., Murphy, P. V., Oscarson, S., Roy, R., King, S., et al. (2015). Engineering a therapeutic lectin by uncoupling mitogenicity from antiviral activity. Cell 163, 746-758. 10.1016/j.cell.2015.09.056.
33. Coves-Datson, E. M., King, S. R., Legendre, M., Gupta, A., Chan, S. M., Gitlin, E., Kulkarni, V. V., Pantaleon Garcia, J., Smee, D. F., Lipka, E., Evans, S. E., et al. (2020)). A molecularly engineered antiviral banana lectin inhibits fusion and is efficacious against influenza virus infection in vivo. Proc Natl Acad Sci USA/17, 2122-2132. 10.1073/pnas. 1915152117.
34. Mitchell, C. A., Ramessar, K., and O'Keefe, B. R. (2017). Antiviral lectins: Selective inhibitors of viral entry. Antiviral Res 142, 37-54. 10.1016/j.antiviral.2017.03.007.
35. Barton. C., Kouokam. J. C., Hurst. H., and Palmer. K. E. (2016). Pharmacokinetics of the Antiviral Lectin Griffithsin Administered by Different Routes Indicates Multiple Potential Uses. Viruses 8. 10.3390/v8120331.
36. Zolov. S. N., Rietberg. S. P., and Bonifant. C. L. (2018). Programmed cell death protein 1 activation preferentially inhibits CD28.CAR-T cells. Cytotherapy 20. 1259-1266. 10.1016/j.jcyt.2018.07.005.
37. Eyquem. J., Mansilla-Soto. J., Giavridis. T., van der Stegen, S., Hamieh. M., Cunanan. K. M., Odak. A., Gonen. M., and Sadelain, M. (2017). Targeting a CAR to the TRAC locus with CRISPR/Cas9 enhances tumour rejection. Nature 543. 113-117. 10.1038/nature21405.
38. Long. A. H., Haso. W. M., Shern. J. F., Wanhainen. K. M., Murgai. M., Ingaramo. M., Smith. J. P., Walker. A. J., Kohler. M. E., Venkateshwara. V. R., Kaplan. R. N., et al. (2015). 4-1BB costimulation ameliorates T cell exhaustion induced by tonic signaling of chimeric antigen receptors. Nat Med 21. 581-590. 10.1038/nm.3838.
39. Gheblawi. M., Wang. K., Viveiros. A., Nguyen. Q., Zhong. J. C., Turner, A. J., Raizada. M. K., Grant. M. B., and Oudit. G. Y. (2020)). Angiotensin-Converting Enzyme 2: SARS-CoV-2 Receptor and Regulator of the Renin-Angiotensin System: Celebrating the 20th Anniversary of the Discovery of ACE2. Circ Res 126. 1456-1474. 10.1161/CIRCRESAHA. 120.317015.
40. Crawford. K. H. D., Eguia. R., Dingens. A. S., Loes. A. N., Malone. K. D., Wolf. C. R., Chu. H. Y., Tortorici. M. A., Veesler. D., Murphy. M., Pettie. D., et al. (2020). Protocol and Reagents for Pseudotyping Lentiviral Particles with SARS-CoV-2 Spike Protein for Neutralization Assays. Viruses 12. 10.3390/v12050513.
41. Biron. C. A., Nguyen. K. B., Pien. G. C., Cousens. L. P., and Salazar-Mather. T. P. (1999). Natural killer cells in antiviral defense: function and regulation by innate cytokines. Annu Rev Immunol 17. 189-220. 10.1146/annurev.immunol. 17.1.189.
42. Orange. J. S., Fassett. M. S., Koopman. L. A., Boyson. J. E., and Strominger. J. L. (2002). Viral evasion of natural killer cells. Nat Immunol 3. 1006-1012. 10.1038/ni1102-1006.
43. Taefehshokr. N., Taefehshokr. S., Hemmat. N., and Heit. B. (2020). Covid-19: Perspectives on Innate Immune Evasion. Front Immunol 11. 580641. 10.3389/fimmu.2020.580641.
44. Xu. Y., Liu. Q., Zhong. M., Wang. Z., Chen. Z., Zhang. Y., Xing. H., Tian. Z., Tang. K., Liao. X., Rao. Q., et al. (2019). 2B4 costimulatory domain enhancing cytotoxic ability of anti-CD5 chimeric antigen receptor engineered natural killer cells against T cell malignancies. J Hematol Oncol 12. 49. 10.1186/s13045-019-0732-7.
45. Imai. C., Iwamoto. S., and Campana. D. (2005). Genetic modification of primary natural killer cells overcomes inhibitory signals and induces specific killing of leukemic cells. Blood 106. 376-383. 10.1182/blood-2004-12-4797.
46. Shimasaki. N., Fujisaki. H., Cho. D., Masselli. M., Lockey. T., Eldridge. P., Leung. W., and Campana. D. (2012). A clinically adaptable method to enhance the cytotoxicity of natural killer cells against B-cell malignancies. Cytotherapy 14. 830-840. 10.3109/14653249.2012.671519.
47. Chu, Y., Hochberg, J., Yahr, A., Ayello, J., van de Ven, C., Barth, M., Czuczman, M., and Cairo, M. S. (2015). Targeting CD20+Aggressive B-cell Non-Hodgkin Lymphoma by Anti-CD20 CAR mRNA-Modified Expanded Natural Killer Cells In Vitro and in NSG Mice. Cancer Immunol Res 3, 333-344. 10.1158/2326-6066.CIR-14-0114.
48. Asif Shajahan, Nitin T. Supekar, Anne S. Gleinich, and Azadi, P. (2020). Deducing the N- and O-glycosylation profile of the spike protein of novel coronavirus SARS-CoV-2.
49. Bonifant, C. L., and Tasian, S. K. (2020). The future of cellular immunotherapy for childhood leukemia. Curr Opin Pediatr 32, 13-25. 10.1097/MOP.0000000000000866.
50. Zheng, M., Gao, Y., Wang, G., Song, G., Liu, S., Sun, D., Xu, Y., and Tian, Z. (2020). Functional exhaustion of antiviral lymphocytes in COVID-19 patients. Cell Mol Immunol 17, 533-535. 10.1038/s41423-020-0402-2.
51. Liu, E., Marin, D., Banerjee, P., Macapinlac, H. A., Thompson, P., Basar, R., Nassif Kerbauy, L., Overman, B., Thall, P., Kaplan, M., Nandivada, V., et al. (2020). Use of CAR-Transduced Natural Killer Cells in CD19-Positive Lymphoid Tumors. N Engl J Med 382, 545-553. 10.1056/NEJMoa1910607.
52. Ciurea, S. O., Schafer, J. R., Bassett, R., Denman, C. J., Cao, K., Willis, D., Rondon, G., Chen, J., Soebbing, D., Kaur, I., Gulbis, A., et al. (2017). Phase 1 clinical trial using mbIL21 ex vivo-expanded donor-derived NK cells after haploidentical transplantation. Blood 130, 1857-1868. 10.1182/blood-2017-05-785659.
53. Vera, J., Savoldo, B., Vigouroux, S., Biagi, E., Pule, M., Rossig, C., Wu, J., Heslop, H. E., Rooney, C. M., Brenner, M. K., and Dotti, G. (2006). T lymphocytes redirected against the kappa light chain of human immunoglobulin efficiently kill mature B lymphocyte-derived malignant cells. Blood 108, 3890-3897. 10.1182/blood-2006-04-017061.
54. Bueler, H., and Mulligan, R. C. (1996). Induction of antigen-specific tumor immunity by genetic and cellular vaccines against MAGE: enhanced tumor protection by coexpression of granulocyte-macrophage colony-stimulating factor and B7-1. Mol Med 2. 545-555.
55. Kunz. A., Gern, U., Schmitt, A., Neuber, B., Wang. L., Huckelhoven-Krauss, A., Michels, B., Hofmann, S., Muller-Tidow, C., Dreger, P., Schmitt, M., et al. (2020). Optimized Assessment of qPCR-Based Vector Copy Numbers as a Safety Parameter for GMP-Grade CAR T Cells and Monitoring of Frequency in Patients. Mol Ther Methods Clin Dev 17, 448-454. 10.1016/j.omtm.2020.02.003.
56. Murphy, G. J., Mostoslavsky, G., Kotton, D. N., and Mulligan, R. C. (2006). Exogenous control of mammalian gene expression via modulation of translational termination. Nat Med 12, 1093-1099. 10.1038/nm1376.

### Abbreviations

ACE2, angiotensin-converting enzyme 2
BanLec, Banana Lectin
BL, bioluminescence
CAR, chimeric antigen receptor
CAR-NK, chimeric antigen receptor natural killer
COVID-19, coronavirus disease 2019
E:T, effector-to-target
ffLuc, firefly luciferase
H84T-BanLec, H84T banana lectin
His, histidine
NK, natural killer
RLU, reactive light units
RBD, receptor binding domain
S-protein, spike protein
SARS-CoV-2, severe acute respiratory syndrome coronavirus 2
UTD, untransduced/unmodified
VCN, vector copy number

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All references, e.g., U.S. patents, U.S. patent application publications, PCT patent applications designating the U.S., published foreign patents and patent applications cited herein are incorporated herein by reference in their entireties. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting. While this invention has been particularly shown and described with references to preferred embodiments thereof, it will be understood by those skilled in the art that various changes in form and details may be made therein without departing from the scope of the invention encompassed by the appended claims.

1. An engineered immune cell expressing a chimeric antigen receptor (CAR) polypeptide comprising an extracellular banana lectin (BanLec) domain.

2. The engineered immune cell of claim 1, wherein the BanLec domain comprises the amino acid sequence having at least 70, 75, 80, 85, 90, 91, 92, 93, 94, 95, 96, 97, 98, 99 or 100% identity with an amino acid sequence of SEQ ID NO: 3 or a fragment thereof.

3. The engineered immune cell of claim 1, wherein the BanLec domain comprises the amino acid sequence of SEQ ID NO: 3, or a fragment thereof.

4. The engineered immune cell of claim 1, wherein said BanLec domain is encoded by a polynucleotide sequence having at least 70, 75, 80, 85, 90, 91, 92, 93, 94, 95, 96, 97, 98, 99 or 100% identity with the nucleic acid sequence of SEQ ID NO: 4.

5. The engineered immune cell of claim 1 wherein the BanLec comprises an acid sequence having an H84T substitution.

6. The engineered immune cell of claim 1, wherein the CAR polypeptide comprises the amino acid sequence has at least 70, 75, 80, 85, 90, 91, 92, 93, 94, 95, 96, 97, 98, 99 or 100% identity with an amino acid sequence of SEQ ID NO: 6 OR SEQ ID NO: 7.

7. The engineered immune cell of claim 1, wherein the CAR polypeptide is encoded by a sequence having at least 70, 75, 80, 85, 90, 91, 92, 93, 94, 95, 96, 97, 98, 99 or 100% identity with SEQ ID NO: 8 or SEQ ID NO: 10.

8. (canceled)

9. A method of preventing or treating a viral infection, the method comprising: administering to a subject suffering from or susceptible to a viral infection an effective amount of immune cells of claim 1.

10. A method of preventing or treating a viral infection, the method comprising: providing a composition comprising a population of engineered immune cells of claim 1, administering the composition to the subject, and thereby preventing or treating the viral infection.

11. The method of claim 9, wherein the viral infection comprises a coronavirus infection.

12. The method of claim 9, wherein the viral infection comprises severe acute respiratory syndrome coronavirus 2 (SARS-CoV-2).

13. The method of claim 9, wherein the viral infection comprises a retrovirus infection.

14. (canceled)

15. The method of claim 1, wherein the CAR polypeptide comprises the sequence of SEQ ID NO: 9 or SEQ ID NO: 11.

16. The method of claim 1 wherein the chimeric antigen receptor (CAR) polypeptide comprises an extracellular banana lectin (BanLec) domain, wherein the BanLec domain comprises the amino acid sequence of SEQ ID NO: 3.

17. (canceled)

18. The method of claim 1, further comprising administering an additional, distinct antiviral agent to the subject.

19. (canceled)

20. The method of claim 1, wherein the mammal is a human.

21. (canceled)

22. The method of claim 1, wherein the cells are administered as a pharmaceutical composition comprising a pharmaceutically acceptable carrier.

23. A kit comprising a population of engineered immune cells of claim 1.

24. A method of preventing or treating cells that have been infected by a virus, the method comprising: administering to virally infected cells an effective amount of immune cells of any one of claim 1.

25. A method of treating cells that have been infected by a virus, the method comprising: providing a composition comprising a population of engineered immune cells of claim 1, administering the composition to virally infected cells, and thereby preventing or treating the viral infection in the cells.

26-34. (canceled)


SEQUENCE LISTING (13 sequences)

SEQ ID NO: 1 - protein, 141 aa, Musa acuminata
MNGAIKVGAWGGNGGSAFDMGPAYRIISVKIFSGDVVDGVDVTFTYYGKTETRHYGGSGGTPHEIVLQEGEYLVGMAGEVANYHGAVVLGKLGFSTNKKAYGPFGNTGGTPFSLPIAAGKISGFFGRGGKFLDAIGVYLEP

SEQ ID NO: 2 - DNA, 575 bp, Musa acuminata
atgaacggagcgatcaaggtgggagcatggggagggaacggagggtcggccttcgacatgggacctgcttatcgtatcatcagcgtcaagattttttccggagacgtggtcgacgccgtggacgtcaccttcacctactacgggaagacggagacccgacacttcggtggcagcggtggtactccccacgaggtttgcatcactaccaatctcaaagctcatagctgactgcagattaatggcttctacttggatgcagattgttctgcaggagggcgagtatctggtgggaatgaagggagaatttggtaactaccatggagtggtggtggtggggaagcttggcttcagcaccaacaagaaatcctacggacctttcggcaacacgggagggactcccttctcccttcctatagcagcaggcaagatctctggcttcttcggccgtggcggcgattttattgacgccattggggtctacttggagccataattggccactgcagtaaatcacaagagttgctatgtgctacttggagtgatgagatgaagaatgtctgcaataaatggatcgg

SEQ ID NO: 3 - protein, 141 aa, Artificial Sequence
  Description of Artificial Sequence Synthetic polypeptide
MNGAIKVGAWGGNGGSAFDMGPAYRIISVKIFSGDVVDGVDVTFTYYGKTETRHYGGSGGTPHEIVLQEGEYLVGMAGEVANYTGAVVLGKLGFSTNKKAYGPFGNTGGTPFSLPIAAGKISGFFGRGGKFLDAIGVYLEP

SEQ ID NO: 4 - DNA, 417 bp, Artificial Sequence
  Description of Artificial Sequence Synthetic polynucleotide
aatggcgctatcaaagttggagcctggggcggcaatggcggcagcgcttttgatatgggccctgcctaccggatcatcagcgtgaagatctttagcggcgacgtggtggatggcgtggacgtgacctttacctactacggcaagaccgagacacggcactatggcggaagcggaggaacacctcacgagatcgttctgcaagagggcgagtacctcgttggaatggctggcgaggtggccaactatacaggtgctgtggtgctgggcaagctgggcttcagcaccaacaagaaggcctacggacccttcggcaataccggcggcacaccttttagcctgcctattgccgccggaatcagcggcttttttggcagaggcggcaagttcctggatgccatcggagtgtatctggaaccc

SEQ ID NO: 5 - protein, 139 aa, Artificial Sequence
  Description of Artificial Sequence Synthetic polypeptide
NGAIKVGAWGGNGGSAFDMGPAYRIISVKIFSGDVVDGVDVTFTYYGKTETRHYGGSGGTPHEIVLQEGEYLVGMAGEVANYTGAVVLGKLGFSTNKKAYGPFGNTGGTPFSLPIAAGISGFFGRGGKFLDAIGVYLEP

SEQ ID NO: 6 - protein, 383 aa, Artificial Sequence
  Description of Artificial Sequence Synthetic polypeptide
MDWIWRILFLVGAATGAHSNGAIKVGAWGGNGGSAFDMGPAYRIISVKIFSGDVVDGVDVTFTYYGKTETRHYGGSGGTPHEIVLQEGEYLVGMAGEVANYTGAVVLGKLGFSTNKKAYGPFGNTGGTPFSLPIAAGISGFFGRGGKFLDAIGVYLEPGGGGSTTTPAPRPPTPAPTIASQPLSLRPEACRPAAGGAVHTRGLDFACDIYIWAPAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKRKNPQEGLYNELQKDKMAEAYSEIGMKGERRRGKGHDGLYQGLSTATKDTYDALHMQALPPR

SEQ ID NO: 7 - protein, 434 aa, Artificial Sequence
  Description of Artificial Sequence Synthetic polypeptide
MDWIWRILFLVGAATGAHSNGAIKVGAWGGNGGSAFDMGPAYRIISVKIFSGDVVDGVDVTFTYYGKTETRHYGGSGGTPHEIVLQEGEYLVGMAGEVANYTGAVVLGKLGFSTNKKAYGPFGNTGGTPFSLPIAAGISGFFGRGGKFLDAIGVYLEPGGGGSQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGAGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - DNA, 1305 bp, Artificial Sequence
  Description of Artificial Sequence Synthetic polynucleotide
atggactggatctggcgcatcctgtttcttgtgggagccgccacaggcgcccatagcaatggcgctatcaaagttggagcctggggcggcaatggcggcagcgcttttgatatgggccctgcctaccggatcatcagcgtgaagatctttagcggcgacgtggtggatggcgtggacgtgacctttacctactacggcaagaccgagacacggcactatggcggaagcggaggaacacctcacgagatcgttctgcaagagggcgagtacctcgttggaatggctggcgaggtggccaactatacaggtgctgtggtgctgggcaagctgggcttcagcaccaacaagaaggcctacggacccttcggcaataccggcggcacaccttttagcctgcctattgccgccggaatcagcggcttttttggcagaggcggcaagttcctggatgccatcggagtgtatctggaacccggagggggcggatcccaggattgccagaatgcccaccaagagttccggttctggcccttcctggtcatcatcgtgatcctgagcgccctgttcctgggcaccctggcctgtttttgcgtgtggcgcagaaagcgcaaagagaagcagagcgagacaagccccaaagagttcctgaccatctacgaggacgtgaaggacctgaaaacccggcggaaccacgagcaagagcagacctttcctggcggcggaagcaccatctacagcatgatccagagccagagcagcgcccctacaagccaagagcctgcctacacactgtactccctgatccagcctagcagaaagagcggcagccggaagagaaatcacagccccagcttcaacagcacgatctacgaagtgatcggcaagagccagccaaaggctcagaaccctgccagactgagccggaaagagctggaaaacttcgacgtgtactctggggccggcagagtgaagttcagcagatcagccgatgctcccgcctatcagcagggccagaaccagctgtacaacgagctgaacctggggagaagagaagagtacgacgtgctggacaagcggagaggcagagatcctgagatgggcggaaagccccagcggagaaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatga

SEQ ID NO: 9 - protein, 434 aa, Artificial Sequence; identical to SEQ ID NO: 7
  Description of Artificial Sequence Synthetic polypeptide

SEQ ID NO: 10 - DNA, 1152 bp, Artificial Sequence
  Description of Artificial Sequence Synthetic polynucleotide
atggactggatctggcgcatcctgtttcttgtgggagccgccacaggcgcccatagcaatggcgctatcaaagttggagcctggggcggcaatggcggcagcgcttttgatatgggccctgcctaccggatcatcagcgtgaagatctttagcggcgacgtggtggatggcgtggacgtgacctttacctactacggcaagaccgagacacggcactatggcggaagcggaggaacacctcacgagatcgttctgcaagagggcgagtacctcgttggaatggctggcgaggtggccaactatacaggtgctgtggtgctgggcaagctgggcttcagcaccaacaagaaggcctacggacccttcggcaataccggcggcacaccttttagcctgcctattgccgccggaatcagcggcttttttggcagaggcggcaagttcctggatgccatcggagtgtatctggaacccggagggggcggatccacaacaacccctgcccccagacctcctaccccagcccctacaattgccagccagcctctgagcctgaggcccgaggcttgtagacctgctgctggcggagccgtgcacaccagaggactggatttcgcctgcgacatctacatctgggcccctctggccggcacatgcggagtgctgctgctgagcctcgtgatcaccctgtactgcaagcggggcagaaagaagctgctgtacatcttcaagcagcccttcatgcggcccgtgcagaccacacaggaagaggacggctgctcctgccggttccccgaggaagaagaaggcggctgcgagctgagagtgaagttctctagaagcgccgacgcccctgcctaccagcagggacagaaccagctgtacaacgagctgaacctgggcagacgggaagagtacgacgtgctggacaagcggagaggccgggaccctgagatgggaggcaagagaaagaacccccaggaaggcctgtataacgaactgcagaaagacaagatggccgaggcctacagcgagatcggaatgaagggcgagcggagaagaggcaagggccacgatggactgtatcagggcctgagcaccgccaccaaggacacctatgacgccctgcacatgcaggccctgccccccaga

SEQ ID NO: 11 - protein, 384 aa, Artificial Sequence
  Description of Artificial Sequence Synthetic polypeptide
MDWIWRILFLVGAATGAHSNGAIKVGAWGGNGGSAFDMGPAYRIISVKIFSGDVVDGVDVTFTYYGKTETRHYGGSGGTPHEIVLQEGEYLVGMAGEVANYTGAVVLGKLGFSTNKKAYGPFGNTGGTPFSLPIAAGISGFFGRGGKFLDAIGVYLEPGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKRKNPQEGLYNELQKDKMAEAYSEIGMKGERRRGKGHDGLYQGLSTATKDTYDALHMQALPPR

SEQ ID NO: 12 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - DNA, 29903 bp, Severe acute respiratory syndrome coronavirus 2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa